(19) United States
(12) Patent Application Publication
(10) Pub. No.: US 2025/0282827 A1
(43) Pub. Date: Sep. 11, 2025

(54) LIPIDATED POLYMYXIN ANALOGUES

(71) Applicant: AUCKLAND UNISERVICES LIMITED, Auckland (NZ)

(72) Inventors: Margaret Anne Brimble, Auckland (NZ); Gregory Cook, Dunedin (NZ); Alan James Davidson, Auckland (NZ); Paul William Richard Harris, Auckland (NZ); Veronika Sander, Auckland (NZ)

(21) Appl. No.: 18/682,626

(22) PCT Filed: Aug. 11, 2022
(86) PCT No.: PCT/IB2022/057488

§ 371 Date: Feb. 9, 2024

(30) Foreign Application Priority Data
Aug. 12, 2021 (NZ) 779028

(51) Int. Cl. C07K 7/62; A61K 38/12; A61P 31/04

(52) U.S. Cl. CPC C07K 7/62; A61K 38/12; A61P 31/04

(57) ABSTRACT

The invention relates to polymyxin analogues that contain unique lipids anchored by a novel 2-thioethyl ester linkage via the peptide back bone.

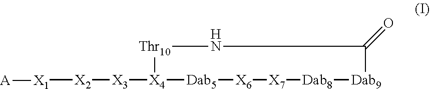

### 1. FIELD OF THE INVENTION

The present invention generally relates to polymyxin analogues that contain unique lipids anchored by a novel 2-thioethyl ester linkage via the peptide back bone.

### 2. BACKGROUND TO THE INVENTION

The world needs need new antibiotics. Since the “golden age” of antibiotic discovery (1940-1960), there has been a considerable slowdown in antibiotic research, with only 30 new compounds progressing to clinical use in the last 20 years. This looming antibiotic crisis is further complicated by escalated resistance in some Gram-negative bacteria, such as Escherichia Coli and Pseudomonas aeruginosa, to most current frontline antibiotics. Gram-negative bacterial infections are more complex to treat due to the cellular structure of the pathogens which have a poorly permeable, double-membrane cell envelope. Unfortunately, most of the newly-approved antibiotics are targeted against Gram-positive bacteria and of those remaining in the clinical pipeline, there are very few that target Gram-negative bacteria.

Accordingly, there is a need for new and improved antibiotics that can effectively target additional species and strains of pathogenic bacteria, particularly Gram-negative pathogenic bacteria.

It is an object of the present invention to go at least some way towards meeting this need by providing at least one new and improved antibiotic that can effectively target pathogenic bacteria, particularly Gram-negative pathogenic bacteria; and/or to at least provide the public with a useful choice. Other objects of the invention may become apparent from the following description which is given by way of example only.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

### 3. SUMMARY OF THE INVENTION

The invention relates to novel polymyxin analogues which comprise one or more unique lipids anchored by a novel 2-thioethyl ester linkage via the peptide backbone.

In one aspect the invention relates to a compound of Formula (I) or a pharmaceutically acceptable salt or solvate thereof;

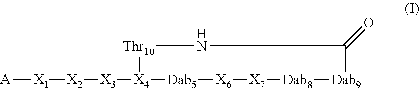

wherein
A is selected from

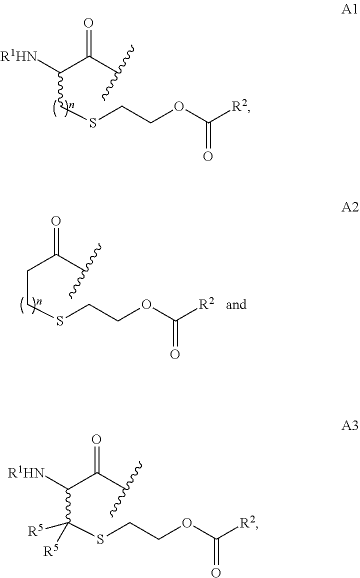

wherein R¹ is H or —C(O)—(C₁-C₁₀)alkyl; n is 1 or 2, and R⁵ is H or CH₃;
X₁ is absent or is Dab;
X₂ is Thr;
X₃ selected from the group consisting of Dab, Dap or D-Ser;
X₄ is selected from the group consisting of Dab, Lys, Orn or Dap;
X₆ is D-Phe or

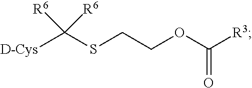

and
X₇ is Leu or

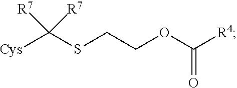

wherein R², R³ and R⁴ are independently selected from the group consisting of —(C₂-C₁₀)alkyl, —(C₃-C₁₀)cycloalkyl, aryl, aryl(C₁-C₁₀)alkyl, —(C₁-C₁₀)alkylaryl, pyridinyl(C₁-C₁₀)alkyl and —(C₁-C₁₀)alkylpyridinyl,
wherein —(C₃-C₁₀)cycloalkyl, aryl and pyridinyl are each independently optionally substituted with halo, —(C₁-C₆)alkyl, —(C₃-C₆)cycloalkyl, —O—(C₁-C₆)alkyl, —O—(C₃-C₆)cycloalkyl, —S(C₁-C₆)alkyl, —S(C₃-C₆)cycloalkyl, —NH(C₁-C₆)alkyl, or NH—(C₃-C₆)cycloalkyl;
and R⁶ and R⁷ are independently selected from H or CH₃.

In another aspect the invention provides a pharmaceutical composition comprising a compound of Formula (I) and a pharmaceutically acceptable carrier.

In another aspect the invention provides a compound of Formula (I) or a pharmaceutically acceptable salt or solvate thereof, for use in treating or preventing a bacterial infection in a subject.

In another aspect the invention provides a method of treating or preventing a bacterial infection in a subject comprising administering to the subject a therapeutically effective amount of a compound of Formula (I) or a pharmaceutically acceptable salt or solvate thereof.

In another aspect the invention provides a use of a compound of Formula (I) or a pharmaceutically acceptable salt or solvate thereof, in the manufacture of a medicament for treating or preventing a bacterial infection in a subject.

In the above aspects, in one embodiment the bacterial infection is a gram-negative bacterial infection.

In another aspect the invention provides a method of killing bacteria comprising contacting the bacteria with a bactericidal amount of a compound of Formula (I) or a pharmaceutically acceptable salt or solvate thereof.

In another aspect the invention provides a method of inhibiting the proliferation of bacteria comprising contacting the bacteria with a bacteriostatic amount of a compound of Formula (I) or a pharmaceutically acceptable salt or solvate thereof.

In the above aspects, in one embodiment the bacteria is a gram-negative bacteria.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Although the present invention is broadly as defined above, those persons skilled in the art will appreciate that the invention is not limited thereto, and that the invention also includes embodiments of which the following description gives examples.

### 4. BRIEF DESCRIPTION OF THE DRAWINGS

The invention will now be described with reference to the accompanying Figures in which:

FIG. 1 is a scheme showing the solid phase synthesis strategy employing S-lipidated building blocks used to prepare S-lipidated analogues of Polymyxin B. Reagents and Conditions: i) iterative Fmoc SPPS, 20% piperidine in DMF (v/v), 2×5 mins, rt then Fmoc-Xaa-OH, HATU, DIPEA, DMF, 20 mins, rt; ii) 20% piperidine in DMF (v/v), 2×5 mins, rt; iii) Building blocks, 3a to 3f, DIPEA, DMF, 1 h, rt; iii) 20% piperidine in DMF, iv) Boc2O, DMF, 2 h; v) 2% N₂H₄·H₂O in DMF (v/v), 3×5 mins, rt; vi) Fmoc-Thr(tBu)-OH, HATU, DIPEA, DMF, 20 mins, vii) Pd(PPh₃)₄, PhSiH₃, CH₂Cl₂:DMF (1:1, v/v), 3 h, rt; viii) 20% piperidine in DMF (v/v), 2×5 mins, rt; ix) PyBOP, HOAt, NMM, DMF, 12 h, rt; x) 90% TFA, 5% TIPS, 5% H₂O (v/v/v), 2 h, rt.

FIG. 2 is a scheme showing the solid phase synthesis strategy for preparing bis S-lipidated polymyxins employed an off resin lipidation strategy. Reagents and Conditions: i) iterative Fmoc SPPS, 20% piperidine in DMF (v/v), 2×5 min, rt then Fmoc-Xaa-OH, HCTU, DIPEA, DMF, 20 mins, rt; ii) 20% piperidine in DMF (v/v), 2×5 min, rt; iii) Boc₂O, DMF, 20 min, rt; iv) NH₂OH·HCl, imidazole, NMP, 5 h, rt; v) Fmoc-Thr(tBu)-OH, HCTU, DIPEA, DMF, 20 mins, rt; vi) Pd(PPh₃)₄, PhSiH3, CH2Cl2, 3 h, rt; vii) 20% piperidine in DMF (v/v), 2×5 mins, rt; viii) PyBOP, HOAt, DIPEA, DMF, 12 h, rt; ix) 94% TFA, 2% TIPS, 2% H₂O, 2% EDT (v/v/v/v), 2 h, rt; x) vinyl ester, DMPA, TIPS, tert-nonanethiol, 5% TFA in NMP (v/v), 1 h, 365 nm, rt; xi) AgOAc, 50% CH₃CN in H₂O (v/v), 12 h, rt then DTT, 50% CH₃CN in H₂O (v/v), 1 h, rt; xii) vinyl ester, DMPA, TIPS, tert-nonanethiol, 5% TFA in NMP (v/v), 1 h, 365 nm, rt

FIG. 3 is a scheme showing an alternative solid phase synthesis of mono S-lipidated polymyxins that vary in the exocyclic portion and the cysteinyl handle Reagents and Conditions: i) iterative Fmoc SPPS, 20% piperidine in DMF (v/v), 2×5 min, rt then Fmoc-Xaa-OH, HATU, DIPEA, DMF, 20 mins, rt; ii) NH₂OH·HCl, imidazole, NMP, 5 h, rt; iii) Alloc-Thr(tBu)-OH, DIC, HOAt, DMF, 16 h; iv) Pd(PPh₃)₄, PhSiH₃, CH₂Cl₂, 3 h; v) PyAOP, HOAt, DIPEA, DMF, 12 h, rt; vi) iterative Fmoc SPPS, 20% piperidine in DMF (v/v), 2×5 min, rt then Fmoc-Xaa-OH, HATU, DIPEA, DMF, 20 mins, rt, for A either Fmoc-Cys(Trt)-OH or Fmoc-D-Cys(Trt)-OH or TrtSCH₂CH₂CO₂H, or homo-L-Cys(Trt)-OH HATU, HOAt, DIPEA, DMF:DCM (1:1), 1 h; viii) 20% piperidine in DMF (v/v), 2×5 min; ix) 95% TFA, 2% TIPS, 1% H₂O, 2% EDT (v/v/v/v), 2 h, rt; x) vinyl propionate, DMPA, TIPS, tert-nonanethiol, 5% TFA in NMP (v/v), 1 h, 365 nm, rt.

FIG. 4 is a scheme showing a chemoenzymatic solution phase synthesis to prepare mono S-lipidated polymyxins. Reagents and Conditions: i) Enzymatic hydrolysis, papain (1.5˜10 unit/mg), DTT, phosphate buffer (0.1 M, pH 6.8), 28 h, 37° C.; ii) Boc-ON, NEt₃, MeOH:H₂O (2:1 v/v), 30 mins, rt; iii) Boc-L-Thz-OH or Boc-D-Thz-OH, HATU, DIPEA, CH₂Cl₂, 2 h, rt; iv) TFA: CH₂Cl₂ (1:1 v/v), 1 h, rt; v) MeONH₂·HCl in H₂O (0.2 M, pH 4), 24 h, rt or 37° C.; vi) vinyl ester, TIPS, tert-nonanethiol, DMPA, 5% TFA in NMP (v/v), 365 nm, 1 h, rt.

FIG. 5 is a graph showing the quantification of apoptosis. Apoptotic (TUNEL+) cells were quantified on paraffin sections of polymyxin-treated organoids (100 μM-1 mM of polymyxin B and compounds 29 and 35). n≥10 organoids per condition. **** p-value ≤0.0001, one-way ANOVA.

FIG. 6 is a graph showing cell survival as a function of the indicated compound concentration. Data are representative of triplicate experiments.

### 5. DETAILED DESCRIPTION OF THE INVENTION

### 5.1 Definitions

The term “comprising” as used in this specification and claims means “consisting at least in part of”. When interpreting each statement in this specification and claims that includes the term “comprising”, features other than that or those prefaced by the term may also be present. Related terms such as “comprise” and “comprises” are to be interpreted in the same manner.

As used herein the term “and/or” means “and” or “or”, or both.

As used herein “(s)” following a noun means the plural and/or singular forms of the noun.

Asymmetric centres may exist in the compounds described herein. The asymmetric centres may be designated as (R) or(S), depending on the configuration of substituents in three-dimensional space at the chiral carbon atom. All chiral, diastereomeric and racemic forms of a structure are intended, unless a particular stereochemistry or isomeric form is indicated. All stereochemical isomeric forms of the compounds, including diastereomeric, enantiomeric, and epimeric forms, as well as d-isomers and l-isomers, and mixtures thereof, including enantiomerically enriched and diastereomerically enriched mixtures of stereochemical isomers, are within the scope of the invention. Therefore, the invention relates to compounds in substantially pure stereoisomeric form with respect to the asymmetric centres of amino acid residues, eg, greater than about 90% de, about 95% to 97% de, or greater than 99% de. Such diastereomers may be prepared by asymmetric synthesis, for example, using chiral intermediates, or mixtures may be resolved using chromatography or other conventional methods.

Individual enantiomers can be prepared synthetically from commercially available enantiopure starting materials or by preparing enantiomeric mixtures and resolving the mixture into individual enantiomers. Resolution methods include (a) separation of an enantiomeric mixture by chromatography on a chiral stationary phase and (b) conversion of the enantiomeric mixture into a mixture of diastereomers and separation of the diastereomers by, for example, recrystallization or chromatography, and any other appropriate methods known in the art. Starting materials of defined stereochemistry may be commercially available or made and, if necessary, resolved by techniques well known in the art. Enantiomers having the “natural” configuration at the chiral carbon (the carbon bearing the CH₂-LG moiety in the seco form) are preferred.

The compounds described herein may also exist as conformational or geometric isomers, including cis, trans, syn, anti, entgegen (E), and zusammen (Z) isomers. All such isomers and any mixtures thereof are within the scope of the invention.

Also within the scope of the invention are any tautomeric isomers or mixtures thereof of the compounds described. As would be appreciated by those skilled in the art, a wide variety of functional groups and other structures may exhibit tautomerism. Examples include, but are not limited to, keto/enol, imine/enamine, and thioketone/enethiol tautomerism.

The compounds described herein may also exist as isotopologues and isotopomers, wherein one or more atoms in the compounds are replaced with different isotopes. Suitable isotopes include, for example, ¹H, ²H (D), ³H (T), ¹²C, ¹³C, ¹⁴C, ¹⁶O, and ¹⁸O. Procedures for incorporating such isotopes into the compounds described herein will be apparent to those skilled in the art. Isotopologues and isotopomers of the compounds described herein are also within the scope of the invention.

Also within the scope of the invention are salts of the compounds described herein, including pharmaceutically acceptable salts. Such salts include, acid addition salts, base addition salts, and quaternary salts of basic nitrogen-containing groups. Acid addition salts can be prepared by reacting compounds, in free base form, with inorganic or organic acids. Examples of inorganic acids include, but are not limited to, hydrochloric, hydrobromic, nitric, sulfuric, and phosphoric acid. Examples of organic acids include, but are not limited to, acetic, trifluoroacetic, propionic, succinic, glycolic, lactic, malic, tartaric, citric, ascorbic, maleic, fumaric, pyruvic, aspartic, glutamic, stearic, salicylic, methanesulfonic, benzenesulfonic, isethionic, sulfanilic, adipic, butyric, and pivalic. Base addition salts can be prepared by reacting compounds, in free acid form, with inorganic or organic bases. Examples of inorganic base addition salts include alkali metal salts, alkaline earth metal salts, and other physiologically acceptable metal salts, for example, aluminium, calcium, lithium, magnesium, potassium, sodium, or zinc salts. Examples of organic base addition salts include amine salts, for example, salts of trimethylamine, diethylamine, ethanolamine, diethanolamine, and ethylenediamine. Quaternary salts of basic nitrogen-containing groups in the compounds may be prepared by, for example, reacting the compounds with alkyl halides such as methyl, ethyl, propyl, and butyl chlorides, bromides, and iodides, dialkyl sulfates such as dimethyl, diethyl, dibutyl, and diamyl sulfates, and the like.

The compounds described herein may form or exist as solvates with various solvents. The term “solvate” as used herein, unless indicated otherwise, refer to an association of one or more solvent molecules and a compound described herein. If the solvent is water, the solvate may be referred to as a hydrate, for example, a monohydrate, a di-hydrate, or a tri-hydrate. All solvated forms and unsolvated forms of the compounds described herein are within the scope of the invention.

The general chemical terms used herein have their usual meanings.

Standard abbreviations for chemical groups are well known in the art and take their usual meaning, eg, Me=methyl, Et=ethyl, Bu=butyl, t-Bu=tert-butyl, Cys=cysteine, Ph=phenyl, Leu=leucine, Phe=phenylalanine, Thr=threonine, Dab=2,4,-diaminobutanoic acid, Dap=2,3-diaminopropionic acid, DMF=dimethylformamide, Orn=2,5-diaminovaleric acid, DIPEA=N,N-diisopropylethylamine, Fmoc=9-fluorenylmethoxycarbonyl, Boc=tert-butoxycarbonyl, HATU=1-[Bis(dimethylamino)methylene]-1H-1,2,3-triazolo[4,5-b]pyridinium 3-oxid hexafluorophosphate, PyBOP=benzotriazol-1-yl-oxytripyrrolidinophosphonium hexafluorophosphate, HOAt=1-Hydroxy-7-azabenzotriazole, NMM=N-methylmorpholine, and TFA=trifluoroacetic acid.

Unless stated otherwise, these abbreviations are applicable to all of the examples below.

The term “alkyl” as used herein alone or in combination with other terms, unless indicated otherwise, refers to a straight-chain or branched saturated or unsaturated acyclic hydrocarbon group. In some embodiments, alkyl groups have from 1 to 15, from 1 to 13, from 1 to 11, from 1 to 10, from 1 to 8, from 1 to 6, from 1 to 5, from 1 to 4, from 1 to 3, from 1 to 2, from 2 to 12, from 2 to 9, from 2 to 8, from 2 to 6, from 2 to 4, from 3 to 9, from 3 to 8, from 4 to 9, from 4 to 15, from 6 to 15, from 8 to 15, from 10 to 15, or 1, or 2, or 3 carbon atoms. In some embodiments, alkyl groups are saturated. Examples of such alkyl groups include but are not limited to -methyl,-ethyl,-n-propyl,-n-butyl,-n-pentyl,-n-hexyl,-n-heptyl,-n-octyl,-n-nonyl,-n-decyl, n-undecyl, n-dodecyl, n-tridecyl, n-tetradecyl, n-pentadecyl,-isopropyl,-sec-butyl,-isobutyl,-tert-butyl,-isopentyl,-neopentyl, 2-methylbutyl,-isohexyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, n-heptyl, isoheptyl, isooctyl, isononyl, isodecyl, isoundecyl, isododecyl, isotridecyl, isotetradecyl, and isopentadecyl and the like. In some embodiments, alkyl groups are unsaturated. Examples of such alkyl groups include but are not limited to -vinyl,-allyl,-1-butenyl,-2-butenyl,-isobutylenyl,-1-pentenyl,-2-pentenyl,-3-methyl-1-butenyl,-2-methyl-2-butenyl,-2,3-dimethyl-2-butenyl, 1-hexyl, 2-hexyl, 3-hexyl,-acetylenyl,-propynyl,-1-butynyl,-2-butynyl,-1-pentynyl,-2-pentynyl,-3-methyl-1-butynyl, and the like. The prefix “Cₓ-C_{y}”, wherein x and y are each an integer, when used in combination with the term “alkyl” refers to the number of carbon atoms in the alkyl group.

The term “cycloalkyl” as used herein alone or in combination with other terms, unless indicated otherwise, refers to a mono or multi-ring (eg, fused, bridged or spiro) non-aromatic hydrocarbon group. In some embodiments, cycloalkyl groups are saturated. The prefix “Cₓ-C_{y}”, wherein x and y are each an integer, when used in combination with the term “cycloalkyl” refers to the number of carbon atoms in the cycloalkyl group.

The term “aryl” as used herein alone or in combination with other terms, unless indicated otherwise, refers to cyclic aromatic hydrocarbon groups that do not contain any ring heteroatoms. Aryl groups include monocyclic, bicyclic and tricyclic ring systems. Examples of aryl groups include, but are not limited to, phenyl, azulenyl, heptalenyl, biphenyl, fluorenyl, phenanthrenyl, anthracenyl, indenyl, indanyl, pentalenyl, and naphthyl. In some embodiments, aryl groups have from 6 to 20, 6 to 14, 6 to 12, or 6 to 10 carbon atoms in the ring(s). In some embodiments, the aryl groups are phenyl or naphthyl. Aryl groups include aromatic-carbocycle fused ring systems. Examples include, but are not limited to, indanyl and tetrahydronaphthyl. The prefix “Cₓ-C_{y}”, wherein x and y are each an integer, when used in combination with the term “aryl” refers to the number of ring carbon atoms in the aryl group. In one embodiment, aryl is phenyl.

The term “pyridinyl” as used herein alone or in combination with other terms, unless indicated otherwise, refers to an aromatic 6-membered ring which comprises one nitrogen atom.

The term “heteroatom” is intended to include oxygen, nitrogen, sulfur, selenium, or phosphorus. In some embodiments, the heteroatom is selected from the group consisting of oxygen, nitrogen, and sulfur.

The term “halo” as used herein alone or in combination with other terms, refers to fluoro, chloro, bromo and iodo, with fluoro, chloro and bromo being preferred and with fluoro and chloro being more preferred.

The term “amino acid” as used herein refers to a molecule containing both an amino group and a carboxyl group bound to a carbon atom which is designated the α-carbon. Amino acids may be naturally occurring or non-naturally occurring. Naturally occurring amino acids include but are not limited to the proteinogenic amino acids known by the one letter abbreviations A, R, N, C, D, Q, E, G, H, I, L, K, M, F, P, S, T, W, Y and W and the three letter abbreviations such as Phe, Leu, Ser, Gly, Cys and the like. Non-naturally occurring amino acids can also form or be included in peptide chains through bonding via their amino and carboxyl groups. Non-naturally occurring amino acids include Dab and Dap and Orn. The present invention contemplates the use of amino acids in both L and D forms, including compounds that incorporate L and D forms of the same amino acid. Unless otherwise indicated, amino acids described for use in the invention are L-amino acids.

The term “pharmaceutically acceptable salt”, as used herein, unless indicated otherwise, refers to pharmaceutically acceptable organic or inorganic salts of the compounds described herein. For example, the compounds described herein may contain an amino group, and accordingly acid addition salts can be formed with this amino group. Examples of salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

The term “therapeutically effective amount” as used herein with reference to an anti-microbial compound or composition as described is an amount that is sufficient to achieve at least a lessening of the symptoms associated with a bacterial infection that is being or is to be treated or that is sufficient to achieve a reduction in bacterial growth, or that is sufficient to increase in bacterial susceptibility to other therapeutic agents or natural immune clearance.

The term “bactericidal amount” as used herein with reference to an anti-microbial compound or composition as described is an amount that is sufficient to kill the bacteria.

The term “bacteriostatic amount” as used herein with reference to an anti-microbial compound or composition as described is an amount that is sufficient to slow the proliferation of the bacteria.

The terms “treatment” and “treating” as used herein cover any treatment of a condition or disease in an animal subject, preferably a mammal, more preferably a human, and includes: (i) inhibiting the bacterial infection, for example, arresting its proliferation; (ii) relieving the bacterial infection, e.g. causing a reduction in the severity of the infection; or (iii) relieving the conditions caused by the bacterial infection, e.g. symptoms of the infection. The terms “prevention” and “preventing” as used herein cover the prevention or prophylaxis of a condition or disease in an animal subject, preferably a mammal, more preferably a human and includes preventing the bacterial infection from occurring in a subject which may be predisposed to infection but has not yet been diagnosed as being infected.

### 5.2 Compounds of the Invention

The polymyxins are a class of cyclic lipopeptides that are potent antimicrobial agents, selective against critical Gram-negative pathogens such as Escherichia Coli, Pseudomonas aeruginosa, Klebsiella pneumoniae and Acinetobacter baumannii. They have been gradually withdrawn from clinical use in the 1970s due to adverse toxicity and the introduction of safer alternatives. However, the polymyxins have since re-emerged as frontline antibiotics as the Gram-negative resistance to known antibiotics has soared.

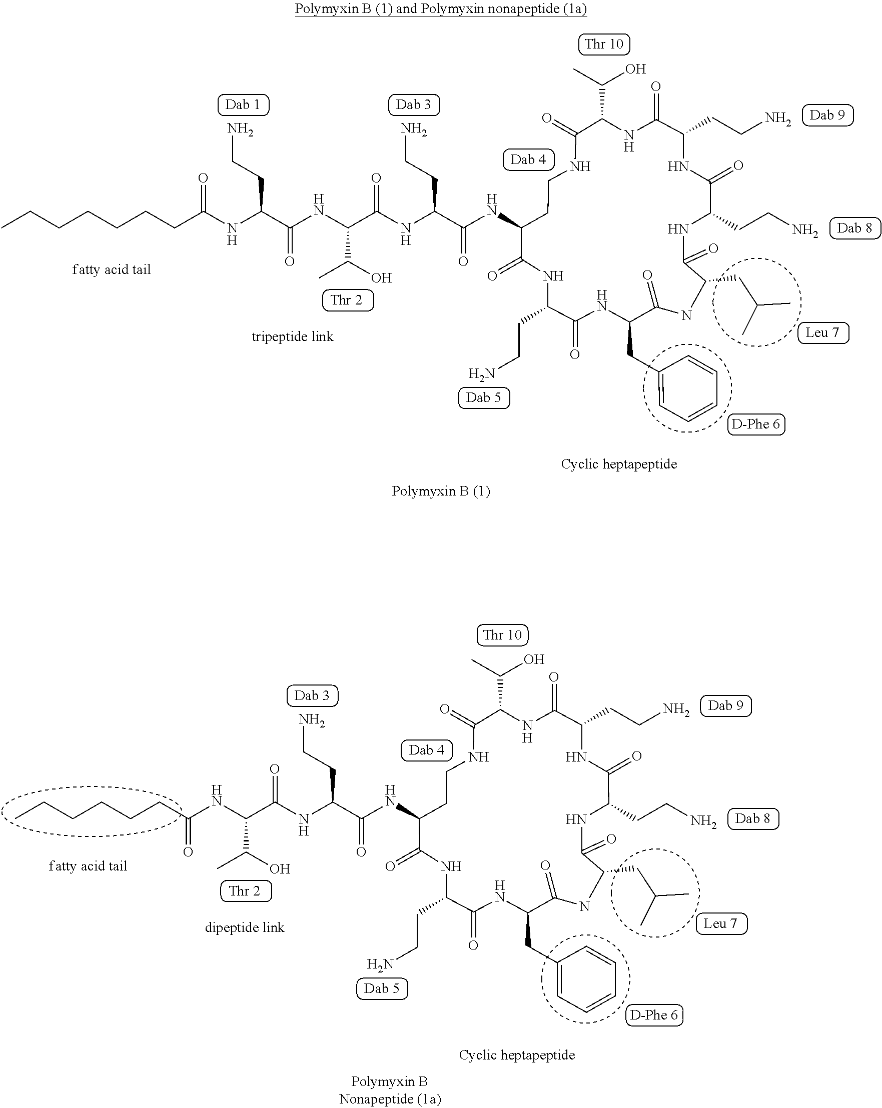

Structurally, the polymyxins consist of a central cyclic heptapeptide core and a linear tri- or dipeptide spacer terminating in an N-acylated lipid (see above, Polymyxin B, 1 and Polymyxin B nonapeptide, 1a). They are defined by the length and branching of the lipid, the hydrophobic amino acids at positions 6 and 7 and the polycationic nature of the non-canonical amino acid 2,4-diaminobutanoic acid (Dab), present in both the cyclic core and the exocyclic tail. Two polymyxins, polymyxin B and polymyxin E (also referred to as Colistin) are in clinical use for multi-drug resistant Gram-negative bacterial infections despite the associated neuro- and nephrotoxicity, and thus require careful administration and renal monitoring.

The inventors have prepared a series of polymyxin B and polymyxin nonapeptide analogues that contain unique lipids anchored by a novel 2-thioethyl ester linkage via the peptide backbone, at either the N-terminus, the 6-position, the 7-position or a combination of several sites. These compounds were found to be similarly active to Polymyxin B with low nephrotoxicity.

Accordingly, in one aspect the invention relates to a compound of Formula (I) or a pharmaceutically acceptable salt or solvate thereof;

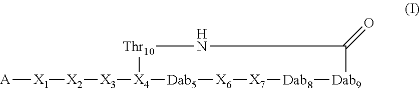

wherein
A is selected from

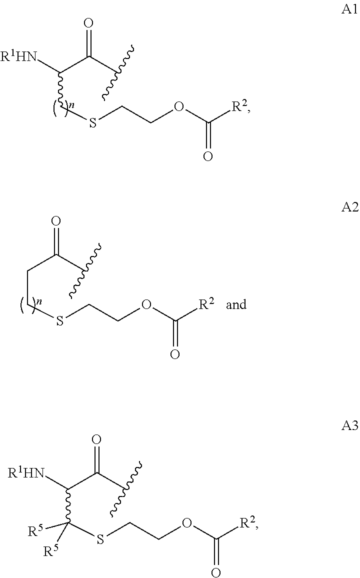

wherein R¹ is H or —C(O)—(C₁-C₁₀)alkyl; n is 1 or 2, and R⁵ is H or CH₃;
X₁ is absent or is Dab;
X₂ is Thr;
X₃ selected from the group consisting of Dab, Dap or D-Ser;
X₄ is selected from the group consisting of Dab, Lys, Orn or Dap;
X₆ is D-Phe or

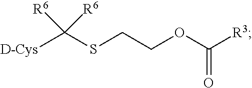

and
X₇ is Leu or

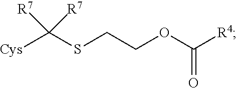

wherein R², R³ and R⁴ are independently selected from the group consisting of —(C₂-C₁₀)alkyl, —(C₃-C₁₀)cycloalkyl, aryl, aryl(C₁-C₁₀)alkyl, —(C₁-C₁₀)alkylaryl, pyridinyl(C₁-C₁₀)alkyl and —(C₁-C₁₀)alkylpyridinyl,
wherein —(C₃-C₁₀)cycloalkyl, aryl and pyridinyl are each independently optionally substituted with halo, —(C₁-C₆)alkyl, —(C₃-C₆)cycloalkyl, —O—(C₁-C₆)alkyl, —O—(C₃-C₆)cycloalkyl, —S(C₁-C₆)alkyl, —S(C₃-C₆)cycloalkyl, —NH(C₁-C₆)alkyl, or NH—(C₃-C₆)cycloalkyl;
and R⁶ and R⁷ are independently selected from H or CH₃.

In one embodiment A is A1. In one embodiment A1 is

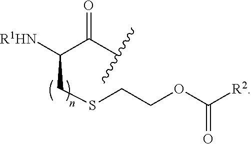

In one embodiment, A1 is

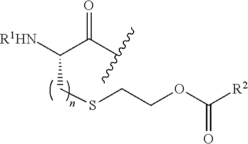

wherein n is 1 and X₁ is Dab.

In one embodiment A1 is

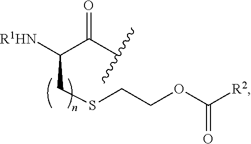

wherein n is 1 and X₁ is absent.

In one embodiment A is A2. In one embodiment, A is A2 wherein n is 1.

In one embodiment A is A3. In one embodiment A3 is

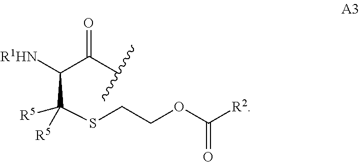

In one embodiment, R² is selected from the group consisting of —(C₂-C₁₀)alkyl, —(C₃-C₁₀)cycloalkyl, aryl, aryl(C₁-C₁₀)alkyl and —(C₁-C₁₀)alkylaryl.

In one embodiment R¹ is H.

In one embodiment X₁ is Dab.

In one embodiment X₃ is Dab.

In one embodiment X₄ is Lys or Dab.

In one embodiment X₆ is D-Phe.

In one embodiment X₇ is Leu.

In one embodiment A is A1 wherein n is 1, and R² is selected from the group consisting of —(C₂-C₁₀)alkyl, aryl and aryl(C₁-C₁₀)alkyl, R¹ is H or —C(O)—(C₁-C₁₀)alkyl, X₁ is Dab, X₂ is Thr, X₃ is Dab, X₄ is Dab or Lys, X₆ is D-Phe or

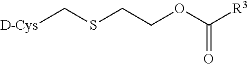

wherein R³ is selected from the group consisting of —(C₂-C₁₀)alkyl, —(C₃-C₁₀)cycloalkyl, aryl, aryl(C₁-C₁₀)alkyl and —(C₁-C₁₀)alkylaryl, preferably —(C₂-C₁₀)alkyl, aryl and aryl(C₁-C₁₀)alkyl and X₇ is Leu or

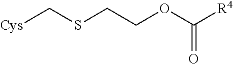

wherein R⁴ is selected from the group consisting of —(C₂-C₁₀)alkyl, —(C₃-C₁₀)cycloalkyl, aryl, aryl(C₁-C₁₀)alkyl and —(C₁-C₁₀)alkylaryl, preferably —(C₂-C₁₀)alkyl, aryl and aryl(C₁-C₁₀)alkyl. Preferably R² is —(C₂-C₁₀)alkyl.

In one embodiment A is A1 wherein n is 1, and R² is selected from the group consisting of —(C₂-C₁₀)alkyl, aryl and aryl(C₁-C₁₀)alkyl, R¹ is H, X₁ is absent, X₃ is Dab, Dap or D-Ser, X₄ is Dab, X₆ is D-Phe and X₇ is Leu. Preferably R² is —(C₂-C₁₀)alkyl.

In one embodiment A is A2 wherein n is 1, and R² is selected from the group consisting of —(C₂-C₁₀)alkyl, aryl and aryl(C₁-C₁₀)alkyl, R¹ is H, X₁ is Dab, X₃ is Dab, X₄ is Dab or Lys, X₆ is D-Phe and X₇ is Leu. Preferably R² is —(C₂-C₁₀)alkyl.

In one embodiment A is A1 wherein n is 2, and R² is selected from the group consisting of —(C₂-C₁₀)alkyl, aryl and aryl(C₁-C₁₀)alkyl, R¹ is H, X₁ is Dab, X₃ is Dab, X₄ is Dab or Lys, X₆ is D-Phe and X₇ is Leu. Preferably R² is —(C₂-C₁₀)alkyl.

In another aspect the invention provides a compound selected from the group consisting of the polymyxin analogues defined in any one of Tables 1 and 3 or a pharmaceutically acceptable salt or solvate thereof.

In one embodiment, the compound of Formula (I) is selected from the group consisting of 29, 44, 59, 60, 76 and 79:

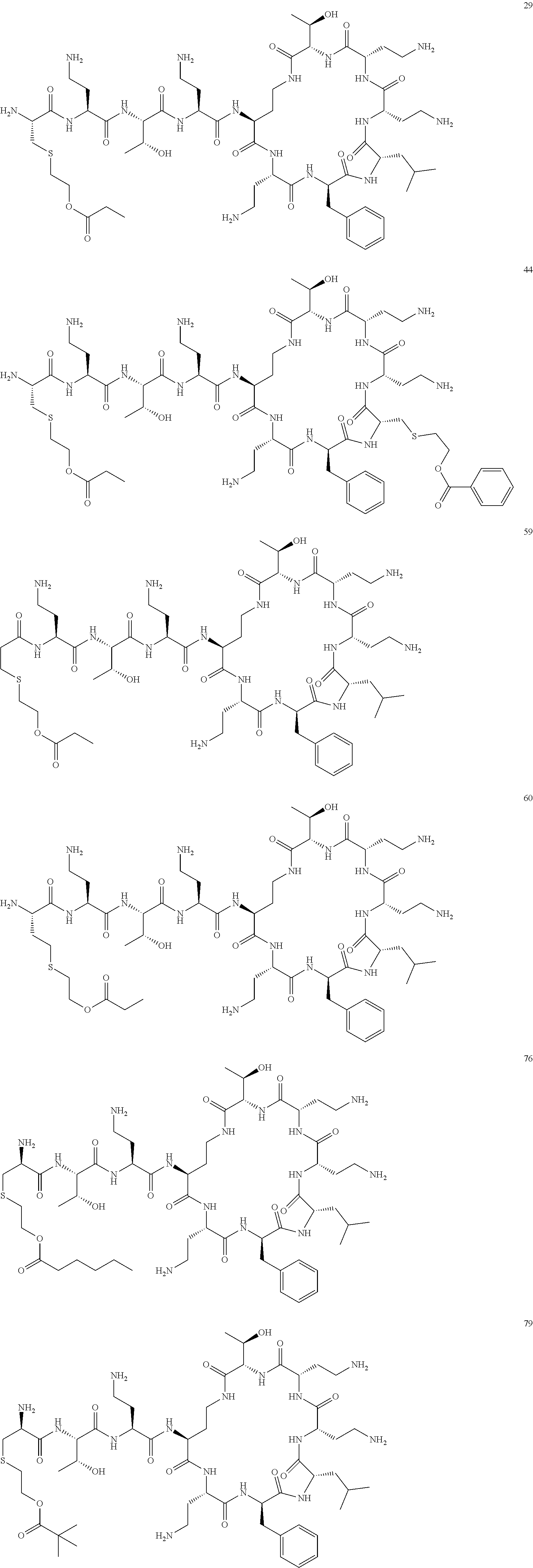

or a pharmaceutically acceptable salt or solvate thereof.

Any of the embodiments of the invention described herein relate to any of the aspects of the invention described herein. The embodiments and preferences described herein may relate alone or in combination to any two or more to any of the aspects of the invention set out herein.

The polymyxin B and polymyxin nonapeptide analogues of the invention may be prepared using solid state peptide synthesis (SSPS). These analogues incorporate into the structure one or more lipidated cysteine residues via a novel a 2-thioethyl ester linkage to the peptide back bone.

The 2-thioethyl ester linkage may be introduced using the inventors' propriety ClipPA (Cysteine Lipidation on a Peptide or Amino acid) technology which utilises a radical-initiated thiol-ene reaction between the free thiol of a cysteine residue and the terminal sp² carbon atom on a fatty acid vinyl ester to prepare lipidated cysteine building blocks (Yang, Harris, Williams, & Brimble, 2016), (Wright, et al., 2013).

The vinyl ester component can be prepared by refluxing the corresponding carboxylic acid vinyl acetate in the presence of mercury acetate (Hg(OAc)₂) and sulfuric acid, as described in Magrone et al. (Magrone, Cavallo, Panzeri, Passarella, & Riva, 2010).

Once prepared, the lipidated cysteine residues are incorporated into the peptide chain making up the polymyxin B or polymyxin nonapeptide analogue.

The lipidated L-cysteine residue at the N-terminus of the compound may be replaced with a cysteine variant such as D-Cys, Des amino-Cys (sulfhydrylpropanoic acid) or homoCys (4-amino-4-sulfanylbutanoic acid).

The polymyxin peptide framework can be prepared using any peptide synthesis technique known in the art. In one embodiment, Fmoc-based SPPS is preferred to assemble the linear peptide, which is then cyclised either in solution or on the solid phase.

The ClipPA technology was used to synthesise a series of novel polymyxin lipopeptide analogues. Details of the synthesis are provided in Examples 1 to 3.

The polymyxin analogues of the invention were screened against E. coli and compared to the known activity profile of synthetic polymyxin B3, as described in Example 4. The nephrotoxicity of selected polymyxin B analogues was measured in Example 5. Their cellular toxicity was evaluated in Example 6. In Example 7, compounds of the invention were tested against a panel of clinically relevant multi-drug resistant Gram-negative pathogens. In Example 8, selected compounds underwent phenotypic antimicrobial susceptibility testing using a panel of clinically relevant multidrug resistant Gram-negative pathogens.

In summary, more than half of the compounds of the invention prepared and tested showed antimicrobial activity with MICs of 1-2 μg/mL against E. coli. Notably, this activity falls within the reported range of both PMB and colistin preparations that are used clinically. Promising activity was also observed against several clinically important pathogen types including carbapenem-resistant Enterobacteriacea and carbapenem-resistant A. baumannii. The dose limitations of the polymyxins due to kidney toxicity is important factor in treating infections. The inventors have surprisingly found that the compounds of the invention generally show little or no toxicity in high doses in a kidney organelle model.

Accordingly, the compounds of the invention constitute new antibiotics with highly desirable properties, that can be utilised for the treatment of microbial infections.

### 5.3 Pharmaceutical Compositions and Uses of the Compounds of the Invention

In another aspect the invention relates to a pharmaceutical composition comprising a compound of Formula (I) or a pharmaceutically acceptable salt or solvate thereof, and a pharmaceutically acceptable carrier. In one embodiment the composition is a pharmaceutical composition.

The term “pharmaceutically acceptable carrier” refers to a carrier, diluent or excipient that may be administered to a subject together with the compound of Formula (I), which is generally safe, non-toxic, and neither biologically nor otherwise undesirable, including carriers suitable for veterinary as well as human pharmaceutical use.

Pharmaceutically acceptable carriers, diluents or excipients that may be used in the compositions include, but are not limited to, ion exchangers, alumina, aluminium stearate, lecithin, self-emulsifying drug delivery systems (SEDDS) such as d-a-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-3-cyclodextrins, or other solubilized derivatives may also be advantageously used to enhance delivery. Oil solutions or suspensions may also contain a long-chain alcohol diluent or dispersant, or carboxymethyl cellulose or similar dispersing agents, which are commonly used in the formulation of pharmaceutically acceptable dosage forms such as emulsions and or suspensions.

In one aspect the invention provides a compound of Formula (I) or a pharmaceutically acceptable salt or solvate thereof, for use in treating or preventing a bacterial infection in a subject.

In one aspect the invention relates to a method of treating or preventing a bacterial infection in a subject comprising administering to the subject a therapeutically effective amount of a compound of Formula (I) or a pharmaceutically acceptable salt or solvate thereof.

In one aspect the invention relates to a use of a compound of Formula (I) or a pharmaceutically acceptable salt or solvate thereof, in the manufacture of a medicament for treating or preventing a bacterial infection in a subject.

In one embodiment the bacterial infection is a Gram-negative bacterial infection.

In some embodiments the Gram-negative bacterial infection may be caused by one or more species selected from one or more of the genera: Acinetobacter; Actinobacillus; Bartonella; Bordetella; Brucella; Burkholderia; Campylobacter; Cyanobacteria; Enterobacter; Envinia; Escherichia; Francisella; Helicobacter; Hemophilus; Klebsiella; Legionella; Moraxella; Morganella; Neisseria; Pasteurella; Proteus; Providencia; Pseudomonas; Salmonella; Serratia; Shigella; Stenotrophomonas; Treponema; Vibrio; and Yersinia.

In particular embodiments, the bacterial infection may be caused by bacteria selected from the group consisting of Pseudomonas aeruginosa, Acinetobacter baumannii, Klebsiella pneumoniae, Klebsiella oxytoca, Stenotrophomonas maltophilia, Enterobacter cloacae, Cifrobacter freundii, Escherichia coli, and Salmonella enterica.

In particular embodiments, the bacterial infection may be caused by bacteria selected from the group consisting of isolates phenotypically characterised as carbapenem-resistant Enterobacteriacea (CRE), extended spectrum β-lactamase (ESBL) producing Enterobacteriacea, colistin-resistant E. coli, carbapenem-resistant Acinetobacter baumannii (CRAB) or carbapenem-resistant Pseudomonas aeruginosa (CRPA).

Use of Polymyxin B and colistin (polymyxin E) have been tested and shown to be effective against some multi-drug resistant (MDR) bacterial infections. However, although they are currently used as a last-line defence in critical cases, observed nephrotoxic side effects have limited their utility.

Nephrotoxicity is also the major dose-limiting factor for the current polymyxins. Therefore, compounds having an improved nephrotoxicity profile would allow higher doses to be administered to more effectively treat infections and suppress the emergence of polymyxin resistance.

Accordingly, in one embodiment, the method comprises administering a compound of Formula (I) or a pharmaceutically acceptable salt or solvate thereof, to a subject for treating or preventing a bacterial infection wherein the compound has a lower nephrotoxicity than polymyxin B and/or colistin. Exemplary methods of assessing nephrotoxicity are described herein and may include other methods known to those of skill in the art.

In further embodiments, the invention provides methods and compounds for sensitizing Gram-negative bacteria to an antibacterial agent or to a host defence mechanism complement present in the serum. The term “sensitizing” as used herein is intended to include any ability to increase the sensitivity, make sensitive or make susceptible a bacterium to an antibacterial agent.

In the therapeutic methods of the invention, the pharmaceutical composition of the invention may be administered as a single dose or in a multiple dose schedule, either as the sole therapeutic agent or simultaneously, sequentially, or separately, in combination with one or more additional therapeutic agents. The one or more additional therapeutic agents will depend on the disease or condition to be treated or other desired therapeutic benefits. The one or more additional therapeutic agents can be used in therapeutic amounts indicated or approved for the particular agent, as would be known to those skilled in the art.

The pharmaceutical compositions are formulated to allow for administration to a subject by any chosen route, including but not limited to oral or parenteral (including topical, subcutaneous, intramuscular and intravenous) administration. In some embodiments, the compositions are formulated for administration orally, intravenously, subcutaneously, intramuscularly, transdermally, intraperitoneally, or other pharmacologically acceptable routes. For example, the compositions may be formulated with an appropriate pharmaceutically acceptable carrier (including excipients, diluents, auxiliaries, and combinations thereof) selected with regard to the intended route of administration and standard pharmaceutical practice. For example, the compositions may be administered orally as a powder, liquid, tablet or capsule, or topically as an ointment, cream or lotion. Suitable formulations may contain additional agents as required, including emulsifying, antioxidant, flavouring or colouring agents, and may be adapted for immediate-, delayed-, modified-, sustained-, pulsed- or controlled-release.

The compositions may be administered via the parenteral route. Examples of parenteral dosage forms include aqueous solutions, isotonic saline or 5% glucose of the active agent, or other well-known pharmaceutically acceptable excipients. Cyclodextrins, for example, or other solubilising agents well-known to those familiar with the art, can be utilized as pharmaceutical excipients for delivery of the therapeutic agent.

Examples of dosage forms suitable for oral administration include, but are not limited to tablets, capsules, lozenges, or like forms, or any liquid forms such as syrups, aqueous solutions, emulsions and the like, capable of providing a therapeutically effective amount of the composition. Capsules can contain any standard pharmaceutically acceptable materials such as gelatin or cellulose. Tablets can be formulated in accordance with conventional procedures by compressing mixtures of the active ingredients with a solid carrier and a lubricant. Examples of solid carriers include starch and sugar bentonite. Active ingredients can also be administered in a form of a hard-shell tablet or a capsule containing a binder, e.g., lactose or mannitol, a conventional filler, and a tabletting agent.

Examples of dosage forms suitable for transdermal administration include, but are not limited, to transdermal patches, transdermal bandages, and the like.

Examples of dosage forms suitable for topical administration of the compositions include any lotion, stick, spray, ointment, paste, cream, gel, etc., whether applied directly to the skin or via an intermediary such as a pad, patch or the like.

Examples of dosage forms suitable for suppository administration of the compositions include any solid dosage form inserted into a bodily orifice particularly those inserted rectally, vaginally and urethrally.

Examples of dosage of forms suitable for injection of the compositions include delivery via bolus such as single or multiple administrations by intravenous injection, subcutaneous, subdermal, and intramuscular administration or oral administration.

Examples of dosage forms suitable for depot administration of the compositions include pellets or solid forms wherein the active(s) are entrapped in a matrix of biodegradable polymers, microemulsions, liposomes or are microencapsulated.

Examples of infusion devices for the compositions include infusion pumps for providing a desired number of doses or steady state administration and include implantable drug pumps. Examples of implantable infusion devices for compositions include any solid form in which the active(s) are encapsulated within or dispersed throughout a biodegradable polymer or synthetic, polymer such as silicone, silicone rubber, silastic or similar polymer.

Examples of dosage forms suitable for transmucosal delivery of the compositions include depositories solutions for enemas, pessaries, tampons, creams, gels, pastes, foams, nebulised solutions, powders and similar formulations containing in addition to the active ingredients such carriers as are known in the art to be appropriate. Such dosage forms include forms suitable for inhalation or insufflation of the compositions, including compositions comprising solutions and/or suspensions in pharmaceutically acceptable, aqueous, or organic solvents, or mixture thereof and/or powders. Transmucosal administration of the compositions may utilize any mucosal membrane but commonly utilizes the nasal, buccal, vaginal and rectal tissues. Formulations suitable for nasal administration of the compositions may be administered in a liquid form, for example, nasal spray, nasal drops, or by aerosol administration by nebulizer, including aqueous or oily solutions of the polymer particles. Formulations may be prepared as aqueous solutions for example in saline, solutions employing benzyl alcohol or other suitable preservatives, absorption promoters to enhance bioavailability, fluorocarbons, and/or other solubilising or dispersing agents known in the art.

Examples of dosage forms suitable for buccal or sublingual administration of the compositions include lozenges, tablets and the like. Examples of dosage forms suitable for ophthalmic administration of the compositions include inserts and/or compositions comprising solutions and/or suspensions in pharmaceutically acceptable, aqueous, or organic solvents.

Examples of formulations of compositions may be found in, for example, Sweetman, S. C. (Ed.). Martindale. The Complete Drug Reference, 33rd Edition, Pharmaceutical Press, Chicago, 2002, 2483 pp.; Aulton, M. E. (Ed.) Pharmaceutics. The Science of Dosage Form Design. Churchill Livingstone, Edinburgh, 2000, 734 pp.; and, Ansel, H. C, Allen, L. V. and Popovich, N. G. Pharmaceutical Dosage Forms and Drug Delivery Systems, 7th Ed., Lippincott 1999, 676 pp. Excipients employed in the manufacture of drug delivery systems are described in various publications known to those skilled in the art including, for example, Kibbe, E. H. Handbook of Pharmaceutical Excipients, 3rd Ed., American Pharmaceutical Association, Washington, 2000, 665 pp. The USP also provides examples of modified-release oral dosage forms, including those formulated as tablets or capsules. See, for example, The United States Pharmacopeia 23/National Formulary 18, The United States Pharmacopeial Convention, Inc., Rockville MD, 1995 (hereinafter “the USP”), which also describes specific tests to determine the drug release capabilities of extended-release and delayed-release tablets and capsules. The USP test for drug release for extended-release and delayed-release articles is based on drug dissolution from the dosage unit against elapsed test time. Descriptions of various test apparatus and procedures may be found in the USP. Further guidance concerning the analysis of extended release dosage forms has been provided by the F.D.A. (See Guidance for Industry. Extended release oral dosage forms: development, evaluation, and application of in vitro/in vivo correlations. Rockville, MD: Center for Drug Evaluation and Research, Food and Drug Administration, 1997).

The dosage forms described herein can be in the form of physically discrete units suitable for use as unitary dosages for the subjects to be treated, each unit containing a predetermined quantity of active material calculated to produce the desired therapeutic effect. Dosage form units may contain from about 0.1 to about 2000 mg of each active ingredient.

Dosage levels of the active ingredients in the pharmaceutical compositions may be varied so as to provide an amount of the active ingredient which is effective to achieve the desired therapeutic effect for a particular patient, composition, and mode of administration, without being toxic to the patient (an effective amount). Data obtained from cell culture assays and animal studies can be used to determine a suitable range of dosage for use in human subjects.

The selected dosage level will depend upon a variety of pharmacokinetic factors including the activity of the particular compositions employed, the route of administration, the time of administration, the rate of excretion of the particular compound of the invention being employed, other drugs, compounds and/or materials used in combination with the particular compositions employed, the age, sex, weight, condition, general health and prior medical history of the patient being treated, and like factors well known in the medical arts. Generally, the daily amount or regimen should be in the range of about 0.01 mg to about 2000 mg of the compound of the invention per kilogram (kg) of body mass.

In another aspect the invention provides a method of killing bacteria comprising contacting the bacteria with a bactericidal amount of a compound of Formula (I) or a pharmaceutically acceptable salt or solvate thereof.

In the context of the present disclosure, “killing” bacteria refers to decrease in the number of viable bacterial cells remaining in a population of bacterial cells exposed to a compound of the invention as described herein as compared to the number of viable bacterial cells in an untreated population. In some embodiments, the “killing” of bacteria is determined by measuring decrease in the number of viable bacterial cells at set time points during culturing in the presence of antibacterial combinations (“time-kill curve”)

In another aspect the invention provides a method of inhibiting the proliferation of at least one bacterial species comprising contacting the bacterial species with a bacteriostatic amount of a compound of Formula (I) or a pharmaceutically acceptable salt or solvate thereof.

In the context of the present disclosure, “inhibiting the proliferation” of at least one bacterial species refers to no detectable increase in the number of bacteria present, and/or in the duration of the bacterial presence or infection under the conditions that otherwise stimulate bacterial multiplication (in the absence of the antibacterial combination).

In some embodiments, “inhibiting the proliferation” of at least one bacterial species is determined by comparative assay of the optical density at 600 nm over time, of a bacterial control culture vs. a bacterial culture treated with an antibacterial combination or composition as described herein. In some embodiments, inhibition is observed when the optical density of the treated culture is less than 10% of the optical density relative to the control culture.

In the above aspects, in one embodiment the bacteria is a gram-negative bacteria.

### 6. EXAMPLES

### General Methods and Materials

All reagents were purchased as reagent grade and used without further purification. Solvents for reactions were distilled prior to use according to standard procedures.

Unless alternative general methods and materials are given, the above general materials and methods are applicable to all the examples below.

### Example 1: Synthesis of Building Blocks for Preparation of Polymyxin B Analogues

Lipidated amino acid building blocks were prepared via a thiol-ene reaction between a cysteine and a vinyl ester (ClipPA), affording an S-lipidated Na-Fmoc amino acid. The use of this reaction to prepare lipidated cysteine residues for incorporation into Fmoc SPPS has been previously described in Yang et al., 2016 and Wright, et al., 2013.

This method was used to prepare six diverse lipidated cysteines, incorporating short or medium chain lipids (3a-3c), a branched lipid (3d) and aromatic lipids (3e and 3f) (Scheme 1).

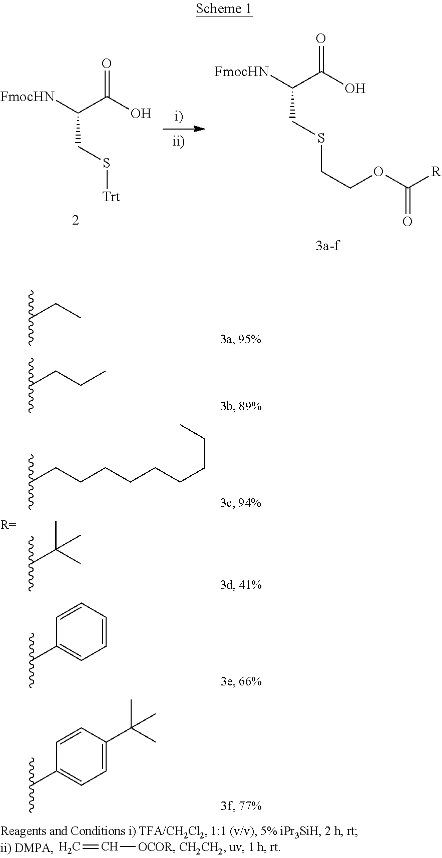

All compounds were obtained in moderate (41%) to excellent (95%) yield and were efficiently purified by standard silica gel flash chromatography to afford the Fmoc-protected S-lipidated cysteines in significant quantities ready for incorporation into SPPS.

### (R)-2-((((9H-Fluoren-9-yl)methoxy)carbonyl)amino-3-((2-(propionyloxy)ethyl)thio)propanoic Acid (3a)

[α]_{D}²¹+14.8 (c 0.014, CHCl₃); ¹H NMR (400 MHZ, CDCl₃): δ_{H}=1.12 (t, J=7.2, 3H, CH₃, H-1), 2.32 (q, J=7.6, 2H, CH₂, H-2), 2.78 (t, J=6.4 Hz, 2H, CH₂, H-6), 3.04-3.17 (m, 2H, CH₂, H-8), 4.23 (t, J=6.8 Hz, 2H, CH₂, H-5), 4.42 (d, J=6.8 Hz, 2H, CH₂, H-13), 4.65 (d, J=6.8 Hz, 1H, CH, H-14), 5.74 (d, J=1.8 Hz, 1H, CH, H-9), 7.31 (t, J=7.2 Hz, 2H, 2×CH, H-17+H-24), 7.39 (t, J=7.6 Hz, 2H, 2×CH, H-18+H-23), 7.60 (d, J=7.6 Hz, 2H, 2×CH, H-16+H-25), 7.75 (d, J=7.6 Hz, 2H, 2×CH, H-19+H-22). ¹³C NMR (100 MHZ, CDCl₃): δ_{C}=9.0 (CH₃, C-1), 27.4 (CH₂, C-2), 31.2 (CH₂, C-6), 34.4 (CH₂, C-8), 47.1 (CH, C-14), 53.6 (CH, C-9), 63.2 (CH₂, C-5), 67.4 (CH₂, C-13), 120.0 (2×CH, C-17+C-24), 127.1 (2×CH, C-18+C-23), 127.8 (2×CH, C-19+C-22), 141.3 (2×C, C-20+C-21), 143.6 (C, C-15), 143.7 (C, C-26), 156.0 (C═O, C-11), 174.6 (C═O, C-3), not observed (C═O, C-27). HRMS-ESI: m/z [M+Na]⁺ calcd for [C₂₃H₂₅NO₆S+Na]⁺ 466.1295. found 466.1297.

### (R)-2-((((9H-Fluoren-9-yl)methoxy)carbonyl)amino-3-((2-(butyryloxy)ethyl)thio)propanoic Acid (3b)

[α]_{D}²¹+8.1 (c 0.019, CHCl₃); ¹H NMR (400 MHZ, CDCl₃): δ_{H}=0.93 (t, J=7.6 Hz, 3H, CH₃, H-1), 1.63 (dq, J=7.2, 14.8 Hz, 2H, CH₂, H-2), 2.29 (t, J=7.6 Hz, 2H, CH₂, H-3), 2.78 (t, J=6.4 Hz, 2H, CH₂, H-7), 3.04-3.22 (m, 2H, CH₂, H-9), 4.23 (t, J=6.8 Hz, 2H, CH₂, H-6), 4.42 (d, J=6.8 Hz, 2H, CH₂, H-14), 4.66 (d, J=6.4 Hz, 1H, CH, H-15), 5.72 (d, J=7.6 Hz, 1H, CH, H-10), 6.07 (BrS, 1H, NH), 7.31 (t, J=7.6 Hz, 2H, 2×CH, H-18+H-25), 7.40 (t, J=7.2, 2H, 2×CH, H-19, H-24), 7.60 (d, J=6.4 Hz, 2H, 2×CH, H-17+H-26), 7.75 (d, J=7.6 Hz, 2H, 2×CH, H-20+H-23). ¹³C NMR (100 MHz, CDCl₃): δ_{C}=13.6 (CH₃, C-1), 18.3 (CH₂, C-2), 31.3 (CH₂, C-7), 34.4 (CH₂, C-9), 36.0 (CH₂, C-3), 47.1 (CH, C-15), 53.5 (CH, C-10), 63.1 (CH₂, C-6), 67.4 (CH₂, C-14), 120.0 (2×CH, C-17+C-26), 125.1 (2×CH, C-18+C-25), 127.1 (2×CH, C-19+C24), 127.8 (2×CH, C-20+C-23), 141.3 (2×C, C-21+C-22), 143.6 (2×C, C-16+C-27), 156.0 (C═O, C-12), 173.8 (C═O, C-4), 174.1 (C═O, C-28). HRMS-ESI: m/z [M+Na]⁺ calcd for [C₂₃H₂₅NO₆S+Na]⁺ 480.1451. found 480.1434.

### (R)-2-((((9H-Fluoren-9-yl)methoxy)carbonyl)amino-3-((2-(undecanoyloxy)ethyl)thio)propanoic Acid (3c)

¹H NMR (400 MHZ, CDCl₃): δ_{H}=0.87 (t, J=6.4 Hz, 4H, CH₃+CH of CH₂, H-1+H-2), 0.93 (t, J=7.2 Hz, 1H, CH of CH₂, H-2), 1.24 (s, 10H, 5×CH₂, H-4+H-5+H-6+H-7+H-8), 1.58-1.64 (m, 2H, CH₂, H-3), 2.29 (t, J=7.2 Hz, 2H, CH₂, H-9), 2.78 (t, J=6.0, 2H, CH₂, H-13), 3.04-3.16 (m, 2H, CH₂, H-15), 4.22 (q, J=6.9 Hz, 2H, CH₂, H-12), 4.42 (d, J=6.8 Hz, 2H, CH₂, H-20), 4.66 (d, J=6.4 Hz, 1H, CH, H-21), 5.54 (BrS, 1H, NH), 5.71 (d, J=7.2 Hz, 1H, CH, H-16), 7.31 (t, J=7.6, 2H, 2×CH, H-24+H-31), 7.40 (t, J=7.6, 2H, 2×CH, H-25+H-30), 7.60 (d, J=6.8 Hz, 2H, 2×CH, H-23+H-32), 7.76 (d, J=7.2 Hz, 2H, 2×CH, H-26+H-29). ¹³C NMR (100 MHZ, CDCl₃): δ_{C}=14.1 (CH₃, C-1), 18.3 (CH₂, C-2), 22.6 (CH₂, C-13), 24.9 (CH₂, C-15), 29.1 (CH₂, C-3), 29.2 (CH₂, C-4), 29.4 (CH₂, C-5), 31.2 (CH₂, C-6), 31.8 (CH₂, C-7), 34.2 (CH₂, C-8), 34.4 (CH₂, C-9), 47.1 (CH, C-21), 53.5 (CH, C-16), 63.1 (CH₂, C-12), 67.4 (CH₂, C-20), 120.0 (2×CH, C-23+C-32), 125.1 (2×CH, C-24+C-31), 127.1 (2×CH, C-25+C-30), 127.8 (2×Ch, C-26+C-29), 141.3 (2×C, C-27+C-28), 143.6 (2×C, C-22+C-33), 155.9 (C═O, C-18), 161.6 (C═O, C-34), 174.1 (C═O, C-10). HRMS-ESI: m/z [M+Na]⁺ calcd for [C₃₀H₃₉NO₆S+Na]⁺ 564.2390. found 564.2381.

### (R)-2-((((9H-Fluoren-9-yl)methoxy)carbonyl)amino-3-((2-(pivaloyloxy)ethyl)thio)propanoic Acid (3d)

[α]_{D}²¹+6.4 (c 0.02, CHCl₃); ¹H NMR (400 MHZ, CDCl₃): δ_{H}=1.19 (s, 9H, 3×CH₃, t-Bu), 2.78 (d, J=2.8 Hz, 2H, CH₂, H-6), 3.04-3.16 (m, 2H, CH₂, H-8), 4.12 (q, J=7.2 Hz, 1H, CH of CH₂, H-5), 4.22 (q, J=7.2 Hz, 1H, CH of CH₂, H-5), 4.41 (d, J=6.8 Hz, 2H, CH₂, H-13), 4.65 (d, J=6.0 Hz, 1H, CH, H-14), 5.76 (d, J=7.6 Hz, 1H, CH, H-9), 7.31 (t, J=7.2 Hz, 2H, 2×CH, H-17+H-24), 7.39 (t, J=7.6 Hz, 2H, 2×CH, H-18+H-23), 7.60 (d, J=6.4 Hz, 2H, 2×CH, H-16+H-25), 7.75 (d, J=7.6 Hz, 2H, 2×CH, H-19+H-22). ¹³C NMR (100 MHZ, CDCl₃): δ_{C}=27.1 (3×CH₃, t-Bu), 31.3 (CH₂, C-6), 34.4 (CH₂, C-8), 38.8 (C, C-2), 47.0 (CH, C-14), 53.6 (CH, C-9), 63.1 (CH₂, C-5), 67.4 (CH₂, C-13), 120.0 (2×CH, C-16+C-22), 125.0 (CH, C-17), 125.1 (CH, C-24), 127.1 (2×CH, C-19+C-22), 127.7 (2×CH, C-18+C-23), 141.3 (2×C, C-30+C-31), 143.6 (C, C-15), 143.7 (C, C-26), 155.9 (C═O, C-11), 174.2 (C═O, C-3), 178.6 (C═O, C-27). HRMS-ESI: m/z [M+Na]⁺ calcd. for [C₃₁H₃₃NO₆S+Na]⁺ 494.1608. found 494.1596.

### (R)-2-((((9H-Fluoren-9-yl)methoxy)carbonyl)amino-3-((2-(benzoyloxy)ethyl)thio)propanoic Acid (3e)

[α]_{D}²¹+9.7 (c 0.01, CHCl₃); ¹H NMR (400 MHZ, CDCl₃): δ_{H}=2.92 (d, J=4.4 Hz, 2H, CH₂, H-10), 3.09-3.21 (m, 2H, CH₂, H-12), 4.22 (t, J=6.8 Hz, 1H, CH of CH₂, H-9), 4.40-4.69 (m, 3H, CH of CH₂+CH₂, H-9+H-17), 4.68 (t, J=4.8, 1H, CH, H-18), 5.23 (BrS, 1H, NH), 5.74 (d, J=7.2, 1H, CH, H-13), 7.29 (t, J=7.2 Hz, 2H, 2×CH, H-22+H-27), 7.38 (t, J=7.2 Hz, 2×CH, H-21+H-28), 7.41 (t, J=7.6 Hz, 2H, 2×CH, H-4+H-2), 7.54 (t, J=7.6 Hz, 1H, H-3), 7.59 (d, J=6.8 Hz, 2H, 2×CH, H-20+H-29), 7.74 (d, J=7.6 Hz, 2H, 2×H, H-23+H-26), 8.02 (d, J=7.2 Hz, 2H, 2×H, H-1+H-5). ¹³C NMR (100 MHZ, CDCl₃): δ_{C}=31.3 (CH₂, C-10), 34.5 (CH₂, C-12), 47.1 (CH, C-18), 53.6 (CH, C-13), 63.7 (CH₂, C-9), 67.3 (CH₂, C-17), 111.0 (2×CH, C-20+C-29), 125.1 (C, C-6), 127.1 (2×CH, C-23+C-26), 127.7 (2×CH, C-22+C-27), 128.4 (2×CH, C-4+C-2), 129.7 (2×CH, C-1+C-5), 133.2 (CH, C-3), 141.3 (2×C, C-24+C-25), 143.6 (2×C, C-19+C-30), 155.9 (C═O, C-15), 171.3 (C═O, C-7), 173.7 (C═O, C-31). HRMS-ESI: m/z [M+Na]⁺ calcd for [C₂₇H₂₅NO₆S+Na]⁺ 514.1295. found 514.1283.

### (R)-2-((((9H-Fluoren-9-yl)methoxy)carbonyl)amino-3-((2-((4-(tert-butyl)benzoyl)oxy)ethyl)thio)propanoic Acid (3f)

[α]_{D}²¹+12.4 (c 0.017, CHCl₃); ¹H NMR (400 MHZ, CDCl₃): δ_{H}=1.31 (s, 9H, 3×CH₃, t-Bu), 2.91 (d, J=5.6 Hz, 2H, CH₂, H-10), 3.16 (dd, J=3.6, 15.6 Hz, 2H, CH₂, H-12), 4.22 (t, J=6.8 Hz, 1H, CH of CH₂, H-9), 4.41-4.47 (m, 3H, CH of CH₂+CH₂, H-9+H-17), 4.68 (d, J=6.4 Hz, 1H, CH, H-18), 5.76 (d, J=7.6 Hz, 1H, CH, H-13), 7.34 (t, J=7.2 Hz, 2H, 2×CH, H-21+H-28), 7.38 (t, J=7.2 Hz, 2H, 2×CH, H-22+H-27), 7.42 (d, J=8.4 Hz, 2H, 2×CH, H-4+H-2), 7.59 (d, J=7.6 Hz, 2H, 2×CH, H-20+H-29), 7.74 (d, J=7.6 Hz, 2H, 2×CH, H-23+H-26), 7.94 (d, J=8.4 Hz, 2H, 2×CH, H-1+H-5). ¹³C NMR (100 MHZ, CDCl₃): δ_{C}=31.0 (3×CH₃, t-Bu), 31.3 (CH₂, C-10), 34.5 (C, C-32), 35.0 (CH₂, C-12), 47.1 (CH, C-18), 53.6 (CH, C-13), 63.6 (CH₂, C-9), 67.3 (CH₂, C-17), 119.9 (2×CH, C-20+C-29), 125.1 (2×CH, C-21+C-28), 125.1 (C, C-6), 125.4 (2×CH, C-23+C-26), 127.0 (2×CH, C-22+C-27), 127.1 (2×CH, C-2+C-4), 129.5 (2×CH, C-1+C-5), 141.2 (2×C, C-24+C-25), 143.7 (2×C, C-19+C-30), 156.8 (C═O, C-15), 166.5 (C═O, C-7), 171.3 (C═O, C-31). HRMS-ESI: m/z [M+Na]⁺ calcd for [C₃₁H₃₃NO₆S+Na]⁺ 570.1921. found 570.1917.

### Example 2: Synthesis of Mono and Bis-S-Lipidated Polymyxins (23 to 60)

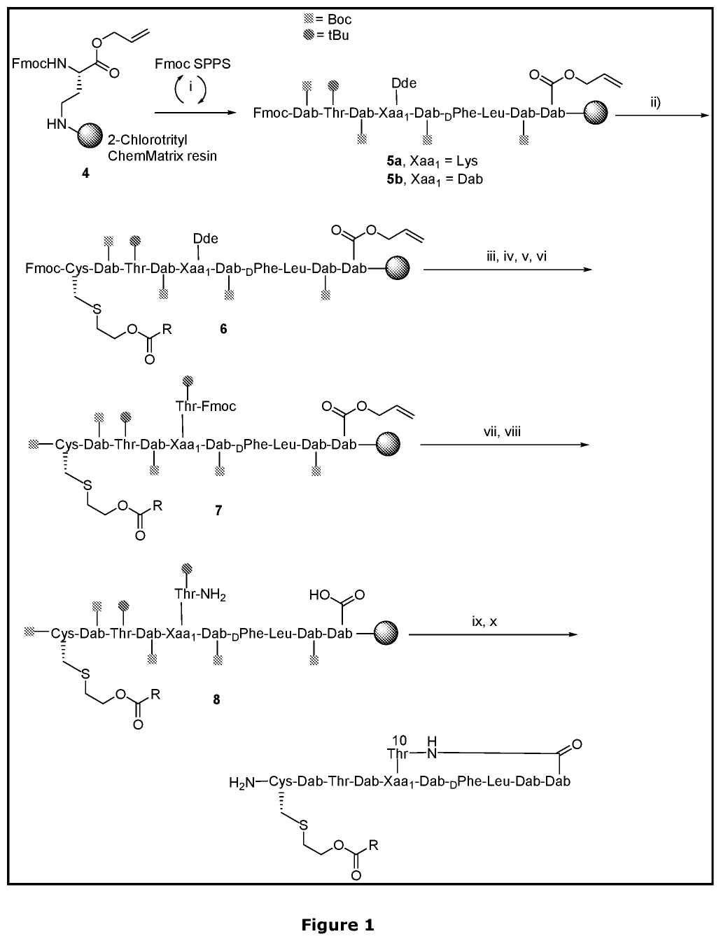

The synthesis of compounds (23-60) was based on a synthesis of polymyxins B2 and E2 (Xu, et al., 2015) and a variant of this synthesis is set out in FIG. 1. The synthesis commenced with functionalisation of 2-chlorotrityl chloride ChemMatrix resin with Fmoc-Dab-O-Allyl via side chain anchoring of the amino group to obtain 4 with a loading of 0.3 mmol/g. Fmoc amino acids were then introduced by standard Fmoc SPPS cycles using 20% piperidine in DMF for Fmoc removal (2×5 mins). Coupling of the incoming Fmoc amino acid, including the lipidated building blocks, was accomplished with HATU/DIPEA for 20 mins (FIG. 1). This afforded resin-bound peptide 5a/5b which was then coupled with the S-lipidated building blocks (3a to 3f) to yield 6. The N-terminal Fmoc protecting group was then replaced with a Boc group to avoid potential side reactions when removing the Dde group from Lys-4/Dab-4 using hydrazine as the Fmoc group is labile to these conditions. Acylation of Lys-4/Dab-4 on the linear resin bound peptide was carried out with Fmoc-Thr(tBu)-OH to afford 7 and the allyl ester on Dab-9 was removed using Pd(PPh₃)₄ in the presence of PhSiH₃ to give 8.

The Fmoc group on Thr-10 was removed by standard Fmoc deblocking conditions and the cyclic peptide was obtained by on-resin macrocyclization using PyBOP and HOAt which was conveniently monitored by the Kaiser test for the absence of free amine. Finally, the free cyclic peptide was released from the resin with concomitant global side chain protecting group removal. The cyclic lipopeptides were recovered, purified by HPLC and the structure confirmed by LC-MS. The yields for the final products were 1.1% to 26% based on the initial resin loading and multi-milligram amounts were obtained in most cases.

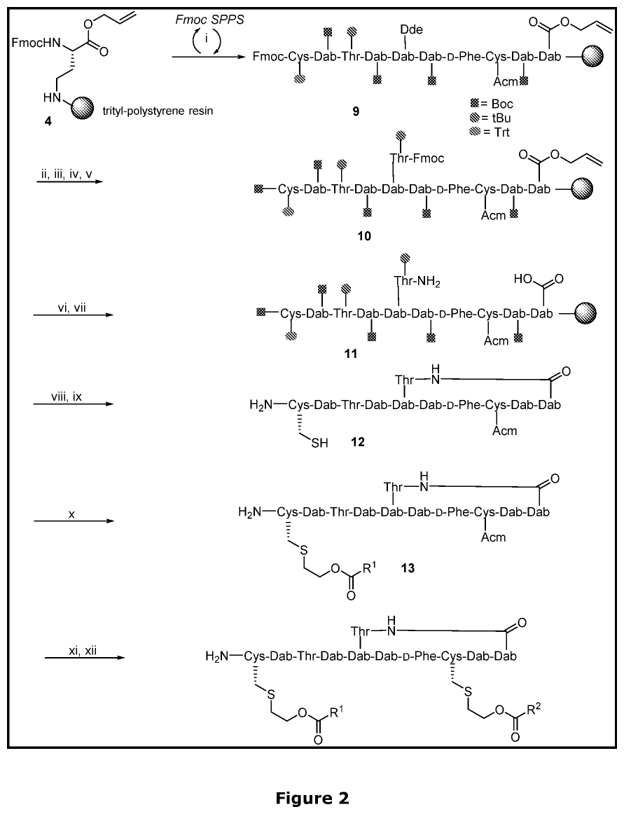

For bis lipidated compounds (35-42 and 49-57), the methodology used was analogous to that depicted in FIG. 1, except that Fmoc-Leu-OH or Fmoc-D-Phe-OH was substituted with the appropriate Fmoc-Cys lipidated building blocks 3a to 3f or their Fmoc-D-Cys lipidated counterparts. The Fmoc-D-Cys lipidated building blocks were prepared according to Example 1 using Fmoc-D-Cys-OH and were characterised by the usual spectroscopic methods. For bis-lipidated compounds (43-48) a procedure employing an orthogonal protecting strategy was employed (FIG. 2). In this variation Fmoc-Cys(Trt)-OH (Trt=triphenymethyl) was used instead of the S-lipidated building blocks at the N-terminus and the Leu-7 was replaced with Fmoc Cys(Acm)-OH (Acm=Acetamidomethyl) to afford resin-bound 9. Replacement of the N-terminal Fmoc group with a Boc, removal of the Dde protecting group on Dab 4 and acylation with Fmoc-Thr(tBu) gave 10. Fmoc removal from Thr-10, allyl deprotection from Dab-9, on resin cyclisation and resin cleavage gave 12. S-Lipidation of the N-terminal cysteine, using standard ClipPA reaction conditions afforded the monolipidated polymyxin 12. The Acm group on Cys-7 was removed with Ag(OAc) to reveal the free thiol, which was S-lipidated yielding the bis-lipidated polymyxins (43-48). Other thiol handles that can be incorporated instead of Cys include those derived from L and D-Cys such as 3-mercapto-D-valine (Penicillamine).

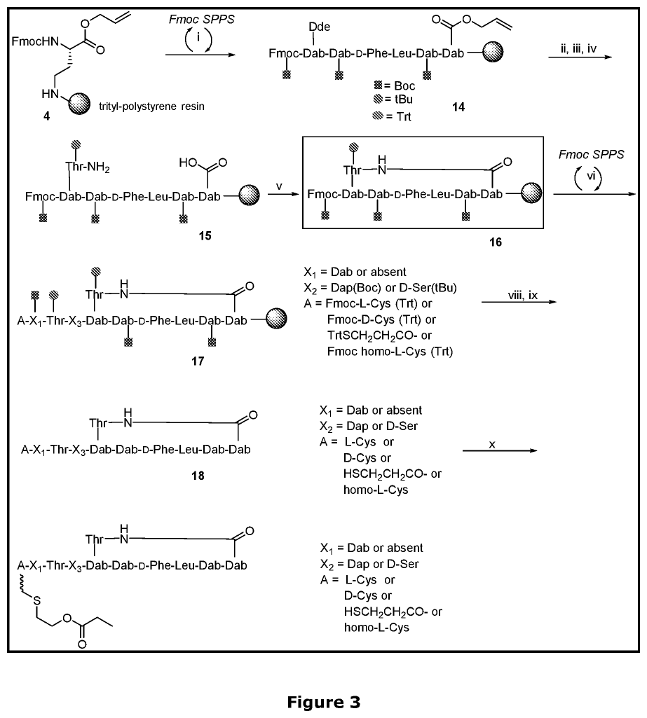

For mono-lipidated polymyxins (58-60) that contained a different thiol handle at the N-terminus and/or different amino acid resides at the exocyclic region (Dab¹-Thr²-Dab³) it was more convenient to prepare a common intermediate 16 as depicted in FIG. 3, incorporate the desired exocyclic amino acids, install the N-terminal thiol-containing amino acid or mercapto acid and then S-lipidate in solution (FIG. 3). Other thiol handles that can be incorporated instead of Cys include those derived from L and D-Cys such as 3-mercapto-D-valine (Penicillamine).

TABLE 1
Structures of Polymyxin B S-lipopeptides
(II)

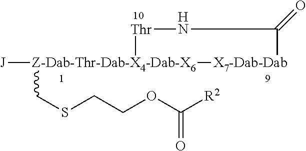

| | | | | |
|---|---|---|---|---|
| 23 | J | R² | Z | X₄ |
| | NH2 | 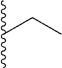 [SMILES: *CC] | L-Cys | Lys |
| | X₆ | X₇ | R³ | R⁴ |
| | D-Phe | Leu | n/a | n/a |
| 24 | J | R² | Z | X₄ |
| | NH₂ | 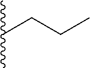 [SMILES: *CCC] | L-Cys | Lys |
| | X₆ | X₇ | R³ | R⁴ |
| | D-Phe | Leu | n/a | n/a |
| 25 | J | R² | Z | X₄ |
| | NH2 | 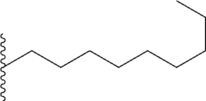 [SMILES: *CCCCCCCCC] | L-Cys | Lys |
| | X₆ | X₇ | R³ | R⁴ |
| | D-Phe | Leu | n/a | n/a |
| 26 | J | R² | Z | X₄ |
| | NH₂ | 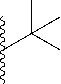 [SMILES: *C(C)(C)C] | L-Cys | Lys |
| | X₆ | X₇ | R³ | R⁴ |
| | D-Phe | Leu | n/a | n/a |
| 27 | J | R² | Z | X₄ |
| | NH₂ | 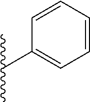 [SMILES: *c1ccccc1] | L-Cys | Lys |
| | X₆ | X₇ | R³ | R⁴ |
| | D-Phe | Leu | n/a | n/a |
| 28 | J | R² | Z | X₄ |
| | NH₂ | 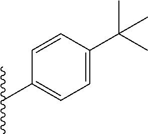 [SMILES: *c1ccc(C(C)(C)C)cc1] | L-Cys | Lys |
| | X₆ | X₇ | R³ | R⁴ |
| | D-Phe | Leu | n/a | n/a |
| 29 | J | R² | Z | X₄ |
| | NH₂ | 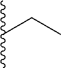 [SMILES: *CC] | L-Cys | Dab |
| | X₆ | X₇ | R³ | R⁴ |
| | D-Phe | Leu | n/a | n/a |
| 30 | J | R² | Z | X₄ |
| | NH₂ | 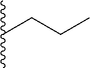 [SMILES: *CCC] | L-Cys | Dab |
| | X₆ | X₇ | R³ | R⁴ |
| | D-Phe | Leu | n/a | n/a |
| 31 | J | R² | Z | X₄ |
| | NH₂ | 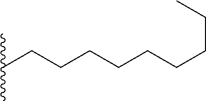 [SMILES: *CCCCCCCCC] | L-Cys | Dab |
| | X₆ | X₇ | R³ | R⁴ |
| | D-Phe | Leu | n/a | n/a |
| 32 | J | R² | Z | X₄ |
| | NH₂ | 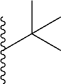 [SMILES: *C(C)(C)C] | L-Cys | Dab |
| | X₆ | X₇ | R³ | R⁴ |
| | D-Phe | Leu | n/a | n/a |
| 33 | J | R² | Z | X₄ |
| | NH₂ | 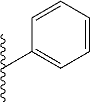 [SMILES: *c1ccccc1] | L-Cys | Dab |
| | X₆ | X₇ | R³ | R⁴ |
| | D-Phe | Leu | n/a | n/a |
| 34 | J | R² | Z | X₄ |
| | NH₂ | 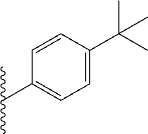 [SMILES: *c1ccc(C(C)(C)C)cc1] | L-Cys | Dab |
| | X₆ | X₇ | R³ | R⁴ |
| | D-Phe | Leu | n/a | n/a |
| 35 | J | R² | Z | X₄ |
| | NH₂ | 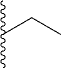 [SMILES: *CC] | L-Cys | Dab |
| | X₆ | X₇ | R³ | R⁴ |
| | D-Phe | 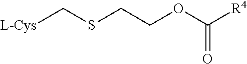 | n/a | 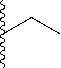 [SMILES: *CC] |
| 36 | J | R² | Z | X₄ |
| | NH₂ | 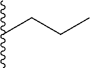 [SMILES: *CCC] | L-Cys | Dab |
| | X₆ | X₇ | R³ | R⁴ |
| | D-Phe | 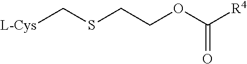 | n/a | 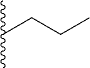 [SMILES: *CCC] |
| 37 | J | R² | Z | X₄ |
| | NH₂ | 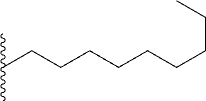 [SMILES: *CCCCCCCCC] | L-Cys | Dab |
| | X₆ | X₇ | R³ | R⁴ |
| | D-Phe | 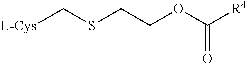 | n/a | 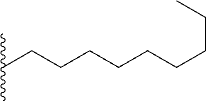 [SMILES: *CCCCCCCCC] |
| 38 | J | R² | Z | X₄ |
| | NH₂ | 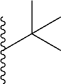 [SMILES: *C(C)(C)C] | L-Cys | Dab |
| | X₆ | X₇ | R³ | R⁴ |
| | D-Phe | 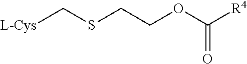 | n/a | 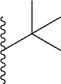 [SMILES: *C(C)(C)C] |
| 39 | J | R² | Z | X₄ |
| | NH₂ | 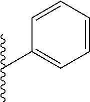 [SMILES: *c1ccccc1] | L-Cys | Dab |
| | X₆ | X₇ | R³ | R⁴ |
| | D-Phe | 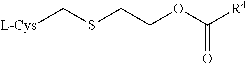 | n/a | 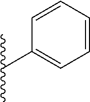 [SMILES: *c1ccccc1] |
| 40 | J | R² | Z | X₄ |
| | NH₂ | 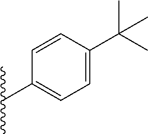 [SMILES: *c1ccc(C(C)(C)C)cc1] | L-Cys | Dab |
| | X₆ | X₇ | R³ | R⁴ |
| | D-Phe | 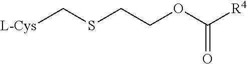 | n/a | 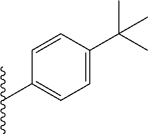 [SMILES: *c1ccc(C(C)(C)C)cc1] |
| 41 | J | R² | Z | X₄ |
| | NH₂ | 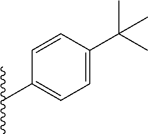 [SMILES: *c1ccc(C(C)(C)C)cc1] | L-Cys | Dab |
| | X₆ | X₇ | R³ | R⁴ |
| | D-Phe | 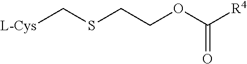 | n/a | 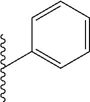 [SMILES: *c1ccccc1] |
| 42 | J | R² | Z | X₄ |
| | NH₂ | 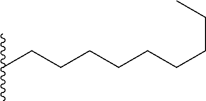 [SMILES: *CCCCCCCCC] | L-Cys | Dab |
| | X₆ | X₇ | R³ | R⁴ |
| | D-Phe | 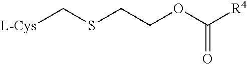 | n/a | 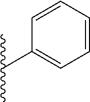 [SMILES: *c1ccccc1] |
| 43 | J | R² | Z | X₄ |
| | NH₂ | 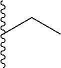 [SMILES: *CC] | L-Cys | Dab |
| | X₆ | X₇ | R³ | R⁴ |
| | D-Phe | 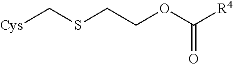 | n/a | 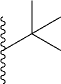 [SMILES: *C(C)(C)C] |
| 44 | J | R² | Z | X₄ |
| | NH₂ | 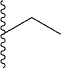 [SMILES: *CC] | L-Cys | Dab |
| | X₆ | X₇ | R³ | R⁴ |
| | D-Phe | 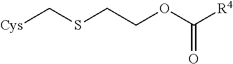 | n/a | 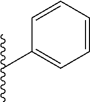 [SMILES: *c1ccccc1] |
| 45 | J | R² | Z | X₄ |
| | NH₂ | 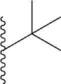 [SMILES: *C(C)(C)C] | L-Cys | Dab |
| | X₆ | X₇ | R³ | R⁴ |
| | D-Phe | 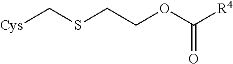 | n/a | 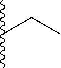 [SMILES: *CC] |
| 46 | J | R² | Z | X₄ |
| | NH₂ | 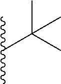 [SMILES: *C(C)(C)C] | L-Cys | Dab |
| | X₆ | X₇ | R³ | R⁴ |
| | D-Phe | 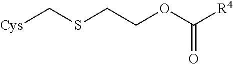 | n/a | 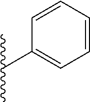 [SMILES: *c1ccccc1] |
| 47 | J | R² | Z | X₄ |
| | NH₂ | 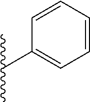 [SMILES: *c1ccccc1] | L-Cys | Dab |
| | X₆ | X₇ | R³ | R⁴ |
| | D-Phe | 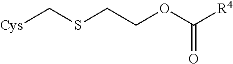 | n/a | 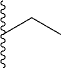 [SMILES: *CC] |
| 48 | J | R² | Z | X₄ |
| | NH₂ | 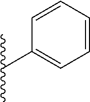 [SMILES: *c1ccccc1] | L-Cys | Dab |
| | X₆ | X₇ | R³ | R⁴ |
| | D-Phe | 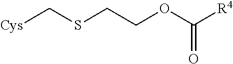 | n/a | 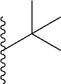 [SMILES: *C(C)(C)C] |
| 49 | J | R² | Z | X₄ |
| | 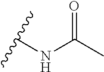 [SMILES: *NC(C)=O] | 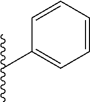 [SMILES: *c1ccccc1] | L-Cys | Dab |
| | X₆ | X₇ | R³ | R⁴ |
| | D-Phe | 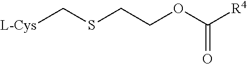 | n/a | 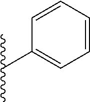 [SMILES: *c1ccccc1] |
| 50 | J | R² | Z | X₄ |
| | 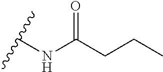 [SMILES: *NC(=O)CCC] | 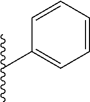 [SMILES: *c1ccccc1] | L-Cys | Dab |
| | X₆ | X₇ | R³ | R⁴ |
| | D-Phe | 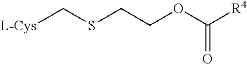 | n/a | 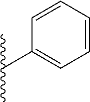 [SMILES: *c1ccccc1] |
| 51 | J | R² | Z | X₄ |
| | 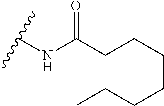 [SMILES: *NC(=O)CCCCCCC] | 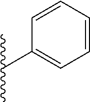 [SMILES: *c1ccccc1] | L-Cys | Dab |
| | X₆ | X₇ | R³ | R⁴ |
| | D-Phe | 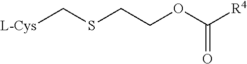 | n/a | 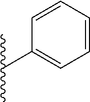 [SMILES: *c1ccccc1] |
| 52 | J | R² | Z | X₄ |
| | 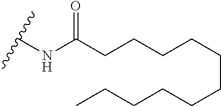 [SMILES: *NC(=O)CCCCCCCCCCC] | 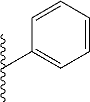 [SMILES: *c1ccccc1] | L-Cys | Dab |
| | X₆ | X₇ | R³ | R⁴ |
| | D-Phe | 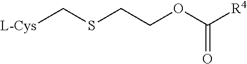 | n/a | 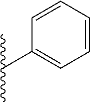 [SMILES: *c1ccccc1] |
| 53 | J | R² | Z | X₄ |
| | NH₂ | 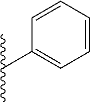 [SMILES: *c1ccccc1] | L-Cys | Dab |
| | X₆ | X₇ | R³ | R⁴ |
| | D-Phe | Leu | n/a | n/a |
| 54 | J | R² | Z | X₄ |
| | NH₂ | 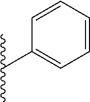 [SMILES: *c1ccccc1] | L-Cys | Dab |
| | X₆ | X₇ | R³ | R⁴ |
| | D-Phe | Leu | n/a | n/a |
| 55 | J | R² | Z | X₄ |
| | NH₂ | 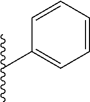 [SMILES: *c1ccccc1] | L-Cys | Dab |
| | X₆ | X₇ | R³ | R⁴ |
| | D-Phe | Leu | n/a | n/a |
| 56 | J | R² | Z | X₄ |
| | NH₂ | 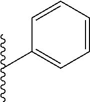 [SMILES: *c1ccccc1] | L-Cys | Dab |
| | X₆ | X₇ | R³ | R⁴ |
| | D-Phe | | n/a | n/a |
| 57 | J | R² | Z | X₄ |
| | NH₂ | 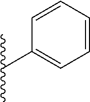 [SMILES: *c1ccccc1] | L-Cys | Dab |
| | X₆ | X₇ | R³ | R⁴ |
| | D-Phe | Leu | n/a | n/a |
| 58 | J | R² | Z | X₄ |
| | NH₂ | 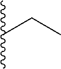 [SMILES: *CC] | D-Cys | Dab |
| | X₆ | X₇ | R³ | R⁴ |
| | D-Phe | Leu | n/a | n/a |
| 59 | J | R² | Z | X₄ |
| | H | 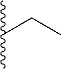 [SMILES: *CC] | MPA | Dab |
| | X₆ | X₇ | R³ | R⁴ |
| | D-Phe | Leu | n/a | n/a |
| 60 | J | R² | Z | X₄ |
| | NH₂ | 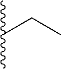 [SMILES: *CC] | h-Cys | Dab |
| | X₆ | X₇ | R³ | R⁴ |
| | D-Phe | Leu | n/a | n/a |

In Table 1, MPA = 2-mercaptoacetyl and h-Cys = homo-L-Cys. In Table 1, the group “Z” refers to the carboxyl group bonded to Dabi and the a-carbon atom to which the side-chain (shown in Formula (II)) is attached. Where Z is “Cys” in Formula (II), A is A1 and n = 1 in Formula (I). The amine group of Cys is depicted as J in Formula (II). Where Z is MPA in Formula (II), A is A2 in Formula (I). Where Z is h-Cys in Formula (II), A is A2, n = 1 in Formula (I). In other words, A in Formula (I) is equivalent to

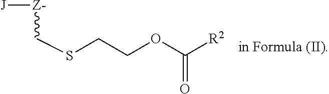

TABLE 2
Mass spectrometry data for S-lipidated polymyxins

| Compound | Mass spectrometry m/z (ESI-MS) |
|---|---|
| 23 | 1295.7 ([M + H]+ requires 1294.72) |
| 24 | 1308.7 ([M + H]+ requires 1308.74) |
| 25 | 1393.8 ([M + H]+ requires 1392.8). |
| 26 | 441.7 [M + 3H]3+ requires 441.9 |
| 27 | 448.4 [M + 3H]3+ requires 448.5 |
| 28 | 467.0 [M + 3H]3+ requires 467.2 |
| 29 | 423.0 [M + 3H]3+ requires 422.7 |
| 30 | 427.6 ([M + 3H]3+ requires 427.8 |
| 31 | 455.9 ([M + 3H]3+ requires 455.9 |
| 32 | 1295.7 ([M + H]+ requires 1294.72). |
| 33 | 1315.6 ([M + H]+ requires 1314.69). |
| 34 | 1371.0 ([M + H]+ requires 1370.75). |
| 35 | 1356.5 ([M + H]+ requires 1356.67). |
| 36 | 1385.6 ([M + H]+ requires 1384.7). |
| 37 | 1553.8 ([M + H]+ requires 1552.89). |
| 38 | 1412.7 ([M + H]+ requires 1412.73). |
| 39 | 1453.7 ([M + H]+ requires 1452.67). |
| 40 | 1564.8 ([M + H]⁺ requires 1564.79). |
| 41 | 754.9 ([M + 2H]2+ requires 754.9). |
| 42 | 1502.4 ([M + H]+ requires 1502.8). |
| 43 | 693.5 ([M + 2H]2+ requires 692.86). |
| 44 | 703.8 ([M + 2H]2+ requires 702.84). |
| 45 | 693.4 ([M + 2H]2+ requires 692.86). |
| 46 | 717.4 ([M + 2H]2+ requires 716.86). |
| 47 | 703.3 ([M + 2H]2+ requires 702.84). |
| 48 | 717.4 ([M + 2H]2+ requires 716.86). |
| 49 | 748.7 ([M + 2H]2+ requires 747.8). |
| 50 | 1522.4 ([M + H]+ requires 1522.7). |
| 51 | 1579.4.([M + H]1 + requires 1578.8). |
| 52 | 1634.4 ([M + H]¹⁺ requires 1634.8). |
| 53 | 1384.8 ([M + H]1+ requires 1384.7). |
| 54 | 735.0 ([M + 2H]2+ requires 735.4). |
| 55 | 1398.8 ([M + H]+ requires 1398.7). |
| 56 | 710.0 ([M + 2H]2+ requires 710.4). |
| 57 | 1474.8 ([M + H]+ requires 1474.8. |
| 58 | 1266.7 ([M + H]+ requires 1265.7). |
| 59 | 1251.9 ([M + H]+ requires 1251.5). |
| 60 | 1280.9 ([M + H]+ requires 1280.6). |

### Example 3: Synthesis of Mono Lipidated Polymyxin Nonapeptides (61 to 104)

Polymyxin nonapeptides are a truncated version of polymyxin B that lack the Dab-1 amino acid. For compounds (61-64 and 81-88) that contained a different thiol handle at the N-terminus and/or different amino acid resides at the exocyclic region (Dab¹-Thr²-Dab³) it was more convenient to prepare a common intermediate 16 as depicted in FIG. 3 as described above for the synthesis of the polymyxins above.

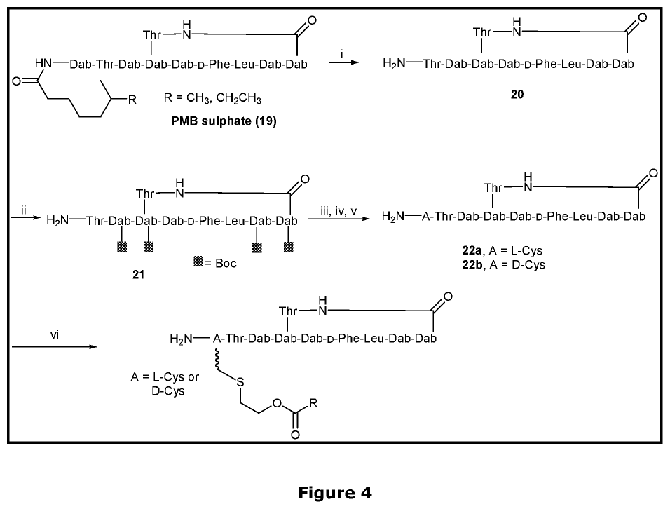

Another synthesis employed for S-lipidated analogues of polymyxin B used a chemoenzymatic approach as reported by Danner et al with modifications (FIG. 4). Briefly, commercially available polymyxin B sulfate was enzymatically delipidated by papain to afford intermediate 20, which was Boc protected on the remaining Dab resides whilst leaving the N-terminal threonine unaffected (compound 21). The cysteinyl thiol handle was introduced onto the peptide at the N-terminus, using a thiazolidine derivative of either L- or D-Cys as this reduced potential racemisation during its introduction. Boc deprotection resulted in cysteinylated polymyxin B nonapeptide 22a/b, that underwent the ClipPA reaction to directly afford S-lipidated polymyxin nonapeptides (65-80, 89-104) containing either an L- or D-Cys handle.

TABLE 3
Structures of Polymyxin B S-lipidated nonapeptides
(III)

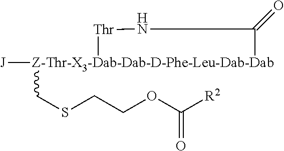

| Compound | J | R² | Z | X₃ |
|---|---|---|---|---|
| 61 | NH₂ | 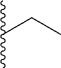 [SMILES: *CC] | L-Cys | Dab |
| 62 | NH₂ | 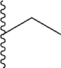 [SMILES: *CC] | D-Cys | Dab |
| 63 | H | 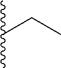 [SMILES: *CC] | MPA | Dab |
| 64 | NH₂ | 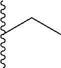 [SMILES: *CC] | h-Cys | Dab |
| 65 | NH₂ | 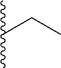 [SMILES: *CC] | L-Cys | Dab |
| 66 | NH₂ | 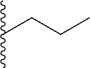 [SMILES: *CCC] | L-Cys | Dab |
| 67 | NH₂ | 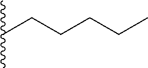 [SMILES: *CCCCC] | L-Cys | Dab |
| 68 | NH₂ | 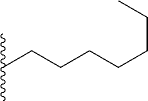 [SMILES: *CCCCCCC] | L-Cys | Dab |
| 69 | NH₂ | 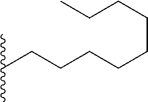 [SMILES: *CCCCCCCCC] | L-Cys | Dab |
| 70 | NH₂ | 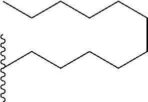 [SMILES: *CCCCCCCCCCC] | L-Cys | Dab |
| 71 | NH₂ | 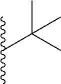 [SMILES: *C(C)(C)C] | L-Cys | Dab |
| 72 | NH₂ | 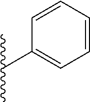 [SMILES: *c1ccccc1] | L-Cys | Dab |
| 73 | NH₂ | 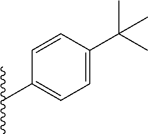 [SMILES: *c1ccc(C(C)(C)C)cc1] | L-Cys | Dab |
| 74 | NH₂ | 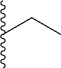 [SMILES: *CC] | D-Cys | Dab |
| 75 | NH₂ | 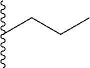 [SMILES: *CCC] | D-Cys | Dab |
| 76 | NH₂ | 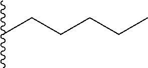 [SMILES: *CCCCC] | D-Cys | Dab |
| 77 | NH₂ | 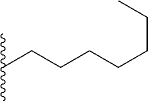 [SMILES: *CCCCCCC] | D-Cys | Dab |
| 78 | NH₂ | 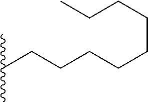 [SMILES: *CCCCCCCCC] | D-Cys | Dab |
| 79 | NH₂ | 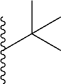 [SMILES: *C(C)(C)C] | D-Cys | Dab |
| 80 | NH₂ | 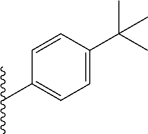 [SMILES: *c1ccc(C(C)(C)C)cc1] | D-Cys | Dab |
| 81 | NH₂ | 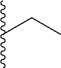 [SMILES: *CC] | L-Cys | Dap |
| 82 | NH₂ | 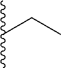 [SMILES: *CC] | D-Cys | Dap |
| 83 | H | 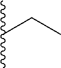 [SMILES: *CC] | MPA | Dap |
| 84 | NH₂ | 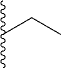 [SMILES: *CC] | h-Cys | Dap |
| 85 | NH₂ | 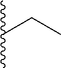 [SMILES: *CC] | L-Cys | D-Ser |
| 86 | NH₂ | 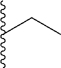 [SMILES: *CC] | D-Cys | D-Ser |
| 87 | H | 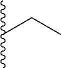 [SMILES: *CC] | MPA | D-Ser |
| 88 | NH₂ | 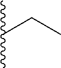 [SMILES: *CC] | h-Cys | D-Ser |
| 89 (TUN160) | NH₂ | 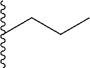 [SMILES: *CCC] | D-Cys | Dab |
| 90 (TUN161) | NH₂ | 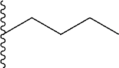 [SMILES: *CCCC] | D-Cys | Dab |
| 91 (TUN134) | NH₂ | 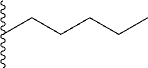 [SMILES: *CCCCC] | D-Cys | Dab |
| 92 (TUN133) | NH₂ | 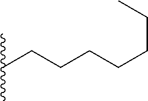 [SMILES: *CCCCCCC] | D-Cys | Dab |
| 93 (TUN138) | NH₂ | 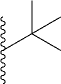 [SMILES: *C(C)(C)C] | D-Cys | Dab |
| 94 (TUN201) | NH₂ | 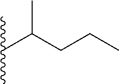 [SMILES: *C(C)CCC] | D-Cys | Dab |
| 95 (TUN235) | NH₂ | 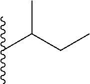 [SMILES: *C(C)CC] | D-Cys | Dab |
| 96 (TUN236) | NH₂ | 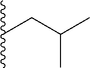 [SMILES: *CC(C)C] | D-Cys | Dab |
| 97 (TUN237) | NH₂ | 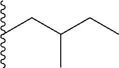 [SMILES: *CC(C)CC] | D-Cys | Dab |
| 98 (TUN238) | NH₂ | 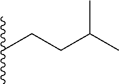 [SMILES: *CCC(C)C] | D-Cys | Dab |
| 99 (TUN245) | NH₂ | 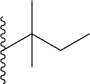 [SMILES: *C(C)(C)CC] | D-Cys | Dab |
| 100 (TUN242) | NH₂ | 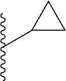 [SMILES: *C1CC1] | D-Cys | Dab |
| 101 (TUN243) | NH₂ | 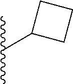 [SMILES: *C1CCC1] | D-Cys | Dab |
| 102 (TUN244) | NH₂ | 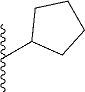 [SMILES: *C1CCCC1] | D-Cys | Dab |
| 103 (TUN234) | NH₂ | 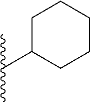 [SMILES: *C1CCCCC1] | D-Cys | Dab |
| 104 (TUN137) | NH₂ | 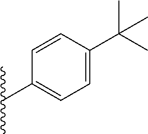 [SMILES: *c1ccc(C(C)(C)C)cc1] | D-Cys | Dab |

In Table 3, MPA = 2-mercaptoacetyl and h-Cys = homo-L-Cys. In Table 3, the group “Z” refers to the carboxyl group bonded to Dabi and the a-carbon atom to which the side-chain (shown in Formula (III)) is attached. Where Z is “Cys” in Formula (III), A is A1 and n = 1 in Formula (I). The amine group of Cys is depicted as J in Formula (III). Where Z is MPA in Formula (III), A is A2 in Formula (I). Where Z is h-Cys in Formula (III), A is A2, n = 1 in Formula (I). In other words, A in Formula (I) is equivalent to

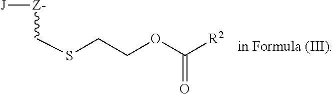

TABLE 4
Mass spectrometry data for S-lipidated polymyxin nonapeptides

| Compound | Mass spectrometry m/z (ESI-MS) |
|---|---|
| 61 | 1166.8([M + H]+ requires 1166.6). |
| 62 | 1166.6 ([M + H]+ requires 1166.6). |
| 63 | 1151.7 ([M + H]+ requires 1151.6). |
| 64 | 1180.5 ([M + H]+ requires 1180.6). |
| 65 | 1166.7 ([M + H]+ requires 1166.6). |
| 66 | 1180.5 ([M + H]+ requires 1180.7). |
| 67 | 1208.6 ([M + H]+ requires 1208.7). |
| 68 | 1236.8 ([M + H]+ requires 1236.7). |
| 69 | 1265.0 ([M + H]+ requires 1264.7). |
| 70 | 1293.0 ([M + H]+ requires 1292.8 |
| 71 | 1194.6 ([M + H]+ requires 1194.7). |
| 72 | 1214.6 ([M + H]+ requires 1214.6). |
| 73 | 1270.6 ([M + H]+ requires 1270.7). |
| 74 | 1166.8 ([M + H]+ requires 1166.6). |
| 75 | 1180.8([M + H]+ requires 1180.7). |
| 76 | 1208.1 ([M + H]+ requires 1208.7). |
| 77 | 1236.8 ([M + H]+ requires 1236.7). |
| 78 | 1265.0 ([M + H]+ requires 1264.7). |
| 79 | 1194.1 ([M + H]+ requires 1194.7). |
| 80 | 1270.9 ([M + H]+ requires 1270.7). |
| 81 | 1152.6 ([M + H]+ requires 1152.6). |
| 82 | 1153.0 ([M + H]+ requires 1152.6). |
| 83 | 1138.0 ([M + H]+ requires 1137.6). |
| 84 | 1166.1 ([M + H]+ requires 1165.3). |
| 85 | 1153.8 ([M + H]+ requires 1153.6). |
| 86 | 1153.8 ([M + H]+ requires 1153.6). |
| 87 | 1138.8 ([M + H]+ requires 1138.6). |
| 88 | 1167.8 ([M + H]+ requires 1167.6). |
| 89 | 1180.1 ([M + H]+ requires 1180.7). |
| (TUN160) | |
| 90 | 1194.2 ([M + H]+ requires 1194.7). |
| (TUN161) | |
| 91 | 1208.1 ([M + H]+ requires 1208.7). |
| (TUN134) | |
| 92 | 1236.8 ([M + H]+ requires 1236.7). |
| (TUN133) | |
| 93 | 1194.1 ([M + H]+ requires 1194.7). |
| (TUN138) | |
| 94 | 1209.0 ([M + H]+ requires 1208.7). |
| (TUN201) | |
| 95 | 1194.6 ([M + H]+ requires 1194.7). |
| (TUN235) | |
| 96 | 1194.9 ([M + H]+ requires 1194.7). |
| (TUN236) | |
| 97 | 1208.8 ([M + H]+ requires 1208.7 |
| (TUN237) | |
| 98 | 1208.9 ([M + H]+ requires 1208.7). |
| (TUN238) | |
| 99 | 1208.9 ([M + H]+ requires 1208.7). |
| (TUN245) | |
| 100 | 1178.7 ([M + H]+ requires 1178.6). |
| (TUN242) | |
| 101 | 1192.8 ([M + H]+ requires 1192.7). |
| (TUN243) | |
| 102 | 1206.9 ([M + H]+ requires 1206.7). |
| (TUN244) | |
| 103 | 1220.8 ([M + H]+ requires 1220.7). |
| (TUN234) | |
| 104 | 1270.9 ([M + H]+ requires 1270.7). |
| (TUN137) | |

### Example 4: Antimicrobial Testing of the Compounds of the Invention

All 81 compounds prepared in Examples 2 and 3 underwent screening against a panel of Gram-negative bacterium to assess their ability to inhibit bacterial growth. This data is presented in Table 5 and was performed in triplicate. The median value is presented.

In general, the majority of S-lipidated polymyxin analogues maintained excellent activity against E. coli and were found to be equipotent to polymyxin B3 (0.25 μg/ml) or within one two fold dilution. Expansion of the polymyxin macrocycle by substituting Dab (29-34)) for Lys (23-28) offered little advantage in antimicrobial activity but was generally well tolerated although for A. baumanni and P. aeruginosa this was detrimental. S-lipidation with short alkyl chains or aromatic groups provided the most potent analogues against most species (26-29, 32-34, 38-40 and 43-48) with 33, 34 and 47 that all contain an aromatic lipid being more potent against the colistin-resistant E. coli ATCC MS8345 than polymyxin.

Analogues designed with S-lipidated cysteine building blocks at the N-terminus alone or the N-terminus and at either position 6 or 7 all provided highly potent analogues, however, position 6 appears less tolerant of certain modifications (53-57). Taking the potent double S-lipidated analogue 39 (0.5 g/ml, E. coli) and introducing acylation to the N-terminus (49-52) proved favourable Variation of the cysteineyl handle (58-60) with the propionyl lipid provided the highly potent compounds, 59 and 60.

In the polymyxin nonapeptide analogues (61-104) a clear preference for a D-Cys at the N-terminus was noted (74-80) as compounds containing an L-Cys (65-73) were mostly inactive. Using an alternative nonapeptide scaffold containing either a Dap (81-84) or D-Ser (85-88) at position 3 with different thiol handles and a propionate lipid resulted in the D-Cys congeners (82 and 86) being most active against E. coli.

Taking the PMB nonapeptide with a D-Cys at the N-terminus, different S-alkyl lipids were examined (89-103). Most retained excellent activity against E. coli, P. aeruginosa and K. pneumoniae but were less potent against A. baumannii and E. coli ATCC MS8345.

TABLE 5
MIC (in μg/mL) of S-lipidated polymyxins and S-lipidated polymyxin nonapeptides
MIC (μg/mL)

| | E. coli | A. baumann | P. aeruginosa | K. pneumoniae | K. pneumoniae | E. coli | K. pneumoniae |
|---|---|---|---|---|---|---|---|
| | ATCC | ii ATCC | ATCC | ATCC | ATCC | ATCC | ATCC |
| Cmpd. | 25922 | 19606 | 27853 | 33495 | 700603 | MS8345 | MS6671 |
| 23 | 4 | >32 | >32 | 4 | >32 | >32 | >32 |
| 24 | 2 | >32 | >32 | 1 | 32->32 | >32 | >32 |
| 25 | 2 | >32 | 0.5 | 0.5 | 2 | 16-32 | >32 |
| 26 | 1 | >32 | >32 | 0.5 | 16 | >32 | >32 |
| 27 | 1 | >32 | >32 | 0.5 | 2-4 | >32 | >32 |
| 28 | 2 | 4 | 1 | 0.5 | 4 | 32 | >32 |
| 29 | 2 | 2 | 2 | 0.125 | 0.5 | 32 | >32 |
| 30 | 0.5 | 2 | 2 | 0.125 | 0.5 | 32 | >32 |
| 31 | 2 | 1 | 1 | 0.5 | 4 | 2-4 | >32 |
| 32 | 0.25 | 1 | 1 | 0.125 | 0.5 | 16 | >32 |
| 33 | 0.25 | 0.5 | 0.5 | 0.0625 | 0.25 | 2 | >32 |
| 34 | 1 | 1 | 0.5 | 0.25 | 1 | 1 | >32 |
| 35 | 0.5 | 32 | >32 | 1-2 | 1 | >32 | >32 |
| 36 | 0.25 | 16 | 16 | 0.25-0.5 | 0.25-0.5 | 32 | >32 |
| 37 | 2-4 | 4 | 4 | 2 | 4 | 4 | >32 |
| 38 | 0.5 | 2 | 4 | 0.5 | 0.5 | 16 | >32 |
| 39 | 0.5 | 2 | 2 | 0.5 | 0.5 | 4 | >32 |
| 40 | 2-4 | 2 | 2 | 2-4 | 2 | 4 | >32 |
| 41 | 1 | 2 | 2 | 2-4 | 1-2 | 4 | >32 |
| 42 | 2 | 2 | 2 | 2-4 | 1-2 | 4 | >32 |
| 43 | 2 | >32 | >32 | 16 | 2-4 | >32 | >32 |
| 44 | 0.5 | 32 | 16 | 0.5 | 0.5 | 32 | >32 |
| 45 | 0.25 | 2-4 | 16 | 0.25-0.5 | 0.25-0.5 | 16-32 | >32 |
| 46 | 2-4 | 16 | 16 | 2 | 1-2 | >32-32 | >32 |
| 47 | 2-4 | >32 | >32 | 2-4 | 2-4 | >32 | >32 |
| 48 | 2 | 32 | 2 | 2-4 | 4 | >32 | >32 |
| 49 | 0.5 | 1-2 | 1 | 0.5 | 0.5 | 16 | >32 |
| 50 | 0.5 | 1-2 | 1 | 0.5-1 | 1 | 16 | >32 |
| 51 | 2 | 1-2 | 2 | 2 | 2-4 | 2-4 | >32 |
| 52 | 2-4 | 2-4 | 4 | 2-4 | 4 | 2-4 | >32 |
| 53 | 0.5 | 16 | 1 | 0.5-1 | 0.5-1 | 16-32 | >32 |
| 54 | 32->32 | >32 | >32 | 32 | 16-32 | >32 | >32 |
| 55 | 0.5 | 16-32 | 4 | 2 | 2 | 32->32 | >32 |
| 56 | 0.5 | 4 | 4 | 1 | 0.5 | 16 | >32 |
| 57 | 1 | 4 | 2 | 2 | 1-2 | 16 | >32 |
| 58 | 2 | >32 | 2 | 2 | 4 | >32-32 | >32 |
| 59 | 0.5 | 32 | 2 | 0.5 | 1 | >32 | >32 |
| 60 | 0.25 | 2 | 0.5 | 0.25 | 0.25-0.5 | 16 | >32 |
| 61 | >128 | n/d | n/d | n/d | n/d | n/d | n/d |
| 62 | 1 | n/d | n/d | n/d | n/d | n/d | n/d |
| 63 | 8 | n/d | n/d | n/d | n/d | n/d | n/d |
| 64 | >128 | n/d | n/d | n/d | n/d | n/d | n/d |
| 65 | 64 | n/d | n/d | n/d | n/d | n/d | n/d |
| 66 | 16 | n/d | n/d | n/d | n/d | n/d | n/d |
| 67 | 4 | n/d | n/d | n/d | n/d | n/d | n/d |
| 68 | 1 | n/d | n/d | n/d | n/d | n/d | n/d |
| 69 | 2 | n/d | n/d | n/d | n/d | n/d | n/d |
| 70 | 8 | n/d | n/d | n/d | n/d | n/d | n/d |
| 71 | 32 | n/d | n/d | n/d | n/d | n/d | n/d |
| 72 | 8 | n/d | n/d | n/d | n/d | n/d | n/d |
| 73 | 1 | n/d | n/d | n/d | n/d | n/d | n/d |
| 74 | 2 | n/d | n/d | n/d | n/d | n/d | n/d |
| 75 | 1 | n/d | n/d | n/d | n/d | n/d | n/d |
| 76 | 0.125 | n/d | n/d | n/d | n/d | n/d | n/d |
| 77 | 0.25 | n/d | n/d | n/d | n/d | n/d | n/d |
| 78 | 0-5-2 | n/d | n/d | n/d | n/d | n/d | n/d |
| 79 | 0.25 | n/d | n/d | n/d | n/d | n/d | n/d |
| 80 | 0.5 | n/d | n/d | n/d | n/d | n/d | n/d |
| 81 | 16 | n/d | n/d | n/d | n/d | n/d | n/d |
| 82 | 1 | n/d | n/d | n/d | n/d | n/d | n/d |
| 83 | 2 | n/d | n/d | n/d | n/d | n/d | n/d |
| 84 | 32 | n/d | n/d | n/d | n/d | n/d | n/d |
| 85 | >128 | n/d | n/d | n/d | n/d | n/d | n/d |
| 86 | 2 | n/d | n/d | n/d | n/d | n/d | n/d |
| 87 | 16 | n/d | n/d | n/d | n/d | n/d | n/d |
| 88 | >64 | n/d | n/d | n/d | n/d | n/d | n/d |
| 89 | 1 | 4 | 2 | 1 | 1 | 32 | >32 |
| 90 | 4 | >16 | 4 | 2 | 4 | >32 | >32 |
| 91 | 1 | 4 | 2 | 1 | 2 | 16 | >32 |
| 92 | 1 | 2 | 2 | 2 | 2 | 16 | >32 |
| 93 | 1 | 4 | 2 | 1 | 2 | 32 | >32 |
| 94 | 1 | 4 | 2 | 1 | 2 | 16 | >32 |
| 95 | 1 | 4 | 1 | 1 | 2 | 16 | >32 |
| 96 | 0.25 | 4 | 1 | 1 | 1 | 16 | >32 |
| 97 | 1 | 4 | 2 | 1 | 2 | 16 | >32 |
| 98 | 0.5 | 2 | 1 | 1 | 1 | 4 | >32 |
| 99 | 2 | 4 | 4 | 2 | 2 | >32 | >32 |
| 100 | 4 | 16 | 4 | 2 | 4 | >32 | >32 |
| 101 | 1 | 4 | 2 | 1 | 2 | >32 | >32 |
| 102 | 1 | 16 | 2 | 1 | 1 | 16 | >32 |
| 103 | 2 | 4 | 2 | 2 | 2 | 16 | >32 |
| 104 | 2 | 4 | 2 | 2 | 4 | 2 | >32 |
| PMB | 0.25 | 0.5 | 0.25 | 0.5 | 0.25 | 4 | >32 |

### Example 5: Nephrotoxicity of Selected ClipPA Polymyxins

Polymyxin-induced kidney injury remains a major dose-limiting factor and can occur in up to 60% of patients. Mechanistically, polymyxins accumulate in renal tubules and cause apoptosis via mitochondrial damage, endoplasmic reticulum stress, oxidative stress and cell cycle arrest (Azad et al., 2019).

Selected polymyxin analogues were evaluated for cytotoxicity in physiologically relevant human kidney tissue, using kidney organoids derived from human induced pluripotent stem cells (Soo et al. 2018). Monolipidated analogues 29, 31, 33, containing a propyl, decyl or phenyl lipid, dilapidated compounds, 35, 38, 39, containing a propyl, tert-butyl or phenyl, lipid, respectively and the N-capped, di-lipidated (phenyl) 49 were selected.

The control compound polymyxin B and the seven ClipPA analogues were tested by adding a range of concentrations (based on Gallado-Godoy et al. 2016) to the organoids at day 12 of the protocol, shown previously to correspond to optimal maturity of the organoid tissues. As positive control, the organoids were treated with 100 μM cisplatin, a chemotherapeutic drug with severe nephrotoxic side effects on patients and kidney organoids (Table 6).

TABLE 6
Summary of nephrotoxicity of polymyxin
B and new analogues on kidney organoids

| Compound. | Concentration | | Score | compound. | Concentration | | Score |
|---|---|---|---|---|---|---|---|
| 29 | 100 | μM | 1 | 58 | 100 | μM | 1 |
| | 300 | μM | 1 | | 300 | μM | 1 |
| | 1 | mM | 2 | 59 | 100 | μM | 1 |
| | 2 | mM | 2 | | 300 | μM | 1 |
| 31 | 100 | μM | 2 | 60 | 100 | μM | 1 |
| | 300 | μM | 3 | | 300 | μM | 1 |
| | 1 | mM | 4 | 75 | 100 | μM | 1 |
| 33 | 100 | μM | 1 | | 300 | μM | 1 |
| | 300 | μM | 3 | | 1 | mM | 3 |
| | 1 | mM | 4 | 76 | 100 | μM | 1 |
| 35 | 100 | μM | 1 | | 300 | μM | 1 |
| | 300 | μM | 1 | | 1 | mM | 3 |
| | 1 | mM | 2 | 82 | 100 | μM | 1 |
| | 2 | mM | 2 | | 300 | μM | 1 |
| 38 | 100 | μM | 1 | | 1 | mM | 1 |
| | 300 | μM | 3 | 83 | 100 | μM | 1 |
| | 1 | mM | 4 | | 300 | μM | 1 |
| 39 | 100 | μM | 1 | | 1 | mM | 1 |
| | 300 | μM | 3 | 86 | 100 | μM | 1 |
| | 1 | mM | 4 | | 300 | μM | 1 |
| 43 | 100 | μM | 1 | | 1 | mM | 1 |
| | 300 | μM | 2 | 91 | 100 | μM | 1 |
| | 1 | mM | 3 | | 300 | μM | 2 |
| 44 | 100 | μM | 1 | | 1 | mM | 3 |
| | 300 | μM | 2 | 92 | 100 | μM | 1 |
| | 1 | mM | 3 | | 300 | μM | 3 |
| 45 | 100 | μM | 1 | | 1 | mM | 4 |
| | 300 | μM | 1 | 93 | 100 | μM | 1 |
| | 1 | mM | 3 | | 300 | μM | 2 |
| 46 | 100 | μM | 1 | | 1 | mM | 3 |
| | 300 | μM | 3 | 94 | 100 | μM | 1 |
| | 1 | mM | 4 | | 300 | μM | 2 |
| 47 | 100 | μM | 1 | | 1 | mM | 3 |
| | 300 | μM | 2 | 103 | 100 | μM | 1 |
| | 1 | mM | 3 | | 300 | μM | 2 |
| 48 | 100 | μM | 1 | | 1 | mM | 3 |
| | 300 | μM | 2 | 104 | 100 | μM | 1 |
| | 1 | mM | 4 | | 300 | μM | 3 |
| 49 | 100 | μM | 3 | | 1 | mM | 4 |
| | 300 | μM | 3 | PMB | 100 | μM | 2 |
| | 1 | mM | 4 | | 300 | μM | 3 |

| | | |
|---|---|---|
| 1 | mM | 4 |

n.d = not determined.
Scores: 1 = unaffected, 2 = mild deterioration, 3 = severe deterioration, 4 = complete deterioration.
−ve control: water = 1,
+ve control 100 μM cisplatin = 3

Toxicity was scored after 48 h for signs of renal tubule deterioration visible by bright field imaging (not shown). This analysis revealed dose-dependent degradation upon treatment with polymyxin B as well as compounds 31, 33, 38-49, 91-104 whereas organoids treated with compounds 29, 35, 58-60, 82-83 and 86 were largely devoid of tissue damage up to 1 mM.

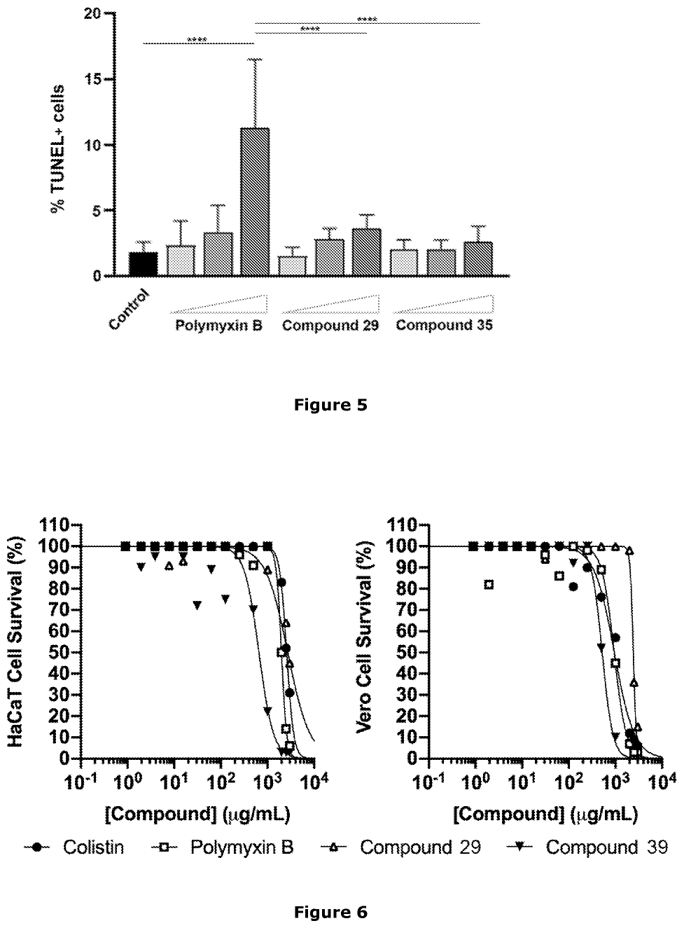

For a quantitative readout of apoptosis, TUNEL+ cells on paraffin sections of compound-treated organoids were measured. A similar trend to that seen in the bright field imaging was observed, i.e., a significantly lower percentage of apoptotic cells in organoids treated with compounds 29 and 35 compared to polymyxin B (FIG. 5).

### Example 7: Cellular Toxicity of Selected ClipPA Polymyxins

To evaluate if the ClipPA modifications introduced any new toxicity, inhibitory concentrations against Vero and HaCaT cell lines were determined. Compound 29 containing the propanoyl lipid was ˜2.5 fold less toxic towards Vero cell lines than either Colistin or Polymyxin B (FIG. 6, and Table 7), while the inhibitory effects against HaCaT cell lines were comparable. The standard error for each measurement is shown.

TABLE 7
Calculated IC₅₀ values for compounds
against Vero or HaCaT cell lines

Cell line IC₅₀ (μg/mL)

| Compound | Vero | HaCaT |
|---|---|---|
| Colistin | 949.7 ± 75.35 | 2578 ± 11.41 |
| Polymyxin B | 939.0 ± 63.32 | 1998 ± 27.74 |
| 29 | 2422 ± 38.98 | 2534 ± 169.2 |
| 39 | 521.0 ± 17.05 | 657.7 ± 87.51 |

Compound 39, bearing phenyl lipids at the N terminus and at Leu-7 was more ˜5 fold more toxic towards both cell lines, compared to compound 29. This is analogous to the obtained nephrotoxicity results where 29 was significantly less toxic. A significant amount of compound 29 was still required to cause inhibition (>500 μg/mL), indicating a low toxicity of polymyxins tested in both cell lines.

### Example 8: Phenotypic Antimicrobial Susceptibility Testing Against Multidrug Resistant Pathogens

Given the promising antimicrobial activity and low toxicity associated with compounds 29, 35 and to a lesser extent 38, these compounds were progressed to a further round of phenotypic antimicrobial susceptibility testing using a panel of clinically relevant multidrug resistant Gram-negative pathogens. This panel included isolates phenotypically characterised as carbapenem-resistant Enterobacteriacea (CRE), extended spectrum β-lactamase (ESBL) producing Enterobacteriacea, colistin-resistant E. coli, carbapenem-resistant Acinetobacter baumannii (CRAB) or carbapenem-resistant Pseudomonas aeruginosa (CRPA). These isolates were chosen because each was resistant to multiple different families of clinically used antibiotics making treatment options limited and importantly, each of these pathogen types are recognised as “Priority 1: CRITICAL” on the World Health Organization (WHO) priority pathogen list for R&D of new antibiotics. Antimicrobial activity against any of these pathogens would therefore be of clinical importance. Encouragingly, each compound displayed promising antimicrobial activity against members of this test panel, with the exceptions being CRPA and colistin-resistant E. coli, which were resistant to all three test compounds and the polymyxin B control (Table 8). However, we note that many of the compounds tested in Example 4 did show good activity against colistin-resistant E. coli.

TABLE 8
MICs of compounds 29, 35 and 38 against clinically
relevant multidrug resistant pathogen types

| Bacterial | | Resistance | MIC (mg/L) | | | |
|---|---|---|---|---|---|---|
| species | Phenotype | mechanism | 29 | 35 | 38 | PMB |
| E. coli | ESBL | CTX-M-15 | 2 | 4 | 2 | 1 |
| E. coli | CRE | NDM-1 | 2 | 2 | 2 | 1 |
| E. cloacae | CRE | IMP-4 | 2 | 2 | 2 | 1 |
| E. coli | CR | MCR-1 | >32 | >32 | 16 | 8 |
| K. pneumoniae | CRE | NDM-5 | 2 | 4 | 2 | 2 |
| K. pneumoniae | CRE | KPC-2 | 2 | 4 | 2 | 2 |
| P. aeruginosa | CRPA | VIM-2 | >32 | >32 | >32 | 8 |
| S. enterica | ESBL | CTX-M-55 | 8 | >32 | 4 | 2 |
| A. baumannii | CRAB | OXA-23 | 4 | 8 | 2 | 1 |

ESBL = extended spectrum β-lactamase producing, CRE = carbapenem-resistant enterobacteriacea, CR = colistin-resistant, CRPA = carbapenem-resistant P. aeruginosa and CRAB = carbapenem-resistant A. baumannii.

Triplicate biological testing revealed minimum inhibitory concentrations within the range 2-8 mg/L for each compound against each pathogen. In almost every case the observed MIC was within 2 doubling dilutions of the MIC observed for the polymyxin B control, clearly demonstrating a similar antimicrobial activity profile to that of polymyxin B for each of the three test compounds.

### 8. REFERENCES

Brown, P., & Dawson, M. J. (2017). Development of new polymyxin derivatives for multi-drug resistant Gram-negative infections. J Antibiot (Tokyo), 70 (4), 386-394.
Danner, R. L., Joiner, K. A., Rubin, M., Patterson, W. H., Johnson, N., Ayers, K. M., & Parrillo, J. E. (1989). Purification, toxicity, and antiendotoxin activity of polymyxin B nonapeptide. Antimicrob. Agents Chemother., 33(9), 1428-1434.
Gallardo-Godoy, A.; Muldoon, C.; Becker, B.; Elliott A. G; Lash L. H; Huang J. X. (2016). Activity and Predicted Nephrotoxicity of Synthetic Antibiotics Based on Polymyxin B. J Med Chem. 59:1068-1077.
Magrone, P., Cavallo, F., Panzeri, W., Passarella, D., & Riva, S. (2010). Exploiting enzymatic regioselectivity: a facile methodology for the synthesis of polyhydroxylated hybrid compounds. Org. Biomol. Chem., 8, 5583-5590.
Rabanal, F., & Cajal, Y. (2017). Recent advances and perspectives in the design and development of polymyxins. Nat. Prod. Rep., 34 (7), 886-908.
Soo, J. Y.; Jansen, J.; Masereeuw, R.; Little, M. (2018) Advances in predictive in vitro models of drug-induced nephrotoxicity. Nat Rev Nephrol. 14, 378-393.
Velkov, T., Roberts, K. D., Nation, R. L., Thompson, P. E., & Li, J. (2013). Pharmacology of polymyxins: new insights into an ‘old’ class of antibiotics. Future Microbiol, 8 (6), 711-724.
Velkov, T., Thompson, P. E., Nation, R. L., & Li, J. (2010). Structure-Activity Relationships of Polymyxin Antibiotics. J. Med. Chem., 53 (5), 1898-1916.
Wright, T. H., Brooks, A. E., Didsbury, A. J., Williams, G. M., Harris, P. W., Dunbar, P. R., & Brimble, M. A. (2013). Direct Peptide Lipidation through Thiol-Ene Coupling Enables Rapid Synthesis and Evaluation of Self-Adjuvanting Vaccine Candidates. Angewandte Chemie-International Edition, 52 (40), 10616-10619.
Xu, W.-L., Cui, A. L., Hu, X.-X., You, X.-F., Li, Z.-R., & Zheng, J.-S. (2015). A new strategy for total solid-phase synthesis of polymyxins. Tetrahedron Lett., 56 (33), 4796-4799.
Yang, S. H., Harris, P. W., Williams, G. M., & Brimble, M. A. (2016). Lipidation of Cysteine or Cysteine-Containing Peptides Using the Thiol-Ene Reaction (CLipPA). Eur. J. Org. Chem., (15), 2608-2616.

1. A compound of Formula (I) or a pharmaceutically acceptable salt or solvate thereof; 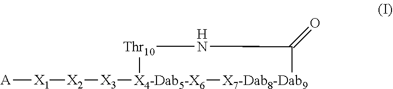
wherein
A is selected from 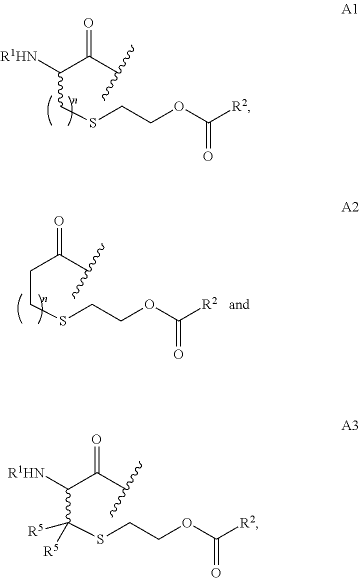
wherein R¹ is H or —C(O)—(C₁-C₁₀)alkyl; n is 1 or 2, and R⁵ is H or CH₃;
X₁ is absent or is Dab;
X₂ is Thr;
X₃ selected from the group consisting of Dab, Dap or D-Ser;
X₄ is selected from the group consisting of Dab, Lys, Orn or Dap;
X₆ is D-Phe or 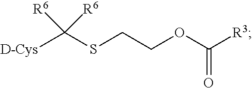
and
X₇ is Leu or 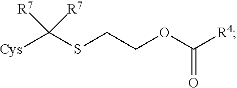
wherein R², R³ and R⁴ are independently selected from the group consisting of —(C₂-C₁₀)alkyl, —(C₃-C₁₀)cycloalkyl, aryl, aryl(C₁-C₁₀)alkyl, —(C₁-C₁₀)alkylaryl, pyridinyl(C₁-C₁₀)alkyl and —(C₁-C₁₀)alkylpyridinyl,
wherein —(C₃-C₁₀)cycloalkyl, aryl and pyridinyl are each independently optionally substituted with halo, —(C₁-C₆)alkyl, —(C₃-C₆)cycloalkyl, —O—(C₁-C₆)alkyl, —O—(C₃-C₆)cycloalkyl, —S(C₁-C₆)alkyl, —S(C₃-C₆)cycloalkyl, —NH(C₁-C₆)alkyl, or NH—(C₃-C₆)cycloalkyl;
and R⁶ and R⁷ are independently selected from H or CH₃.

2. A compound of claim 1 wherein A is 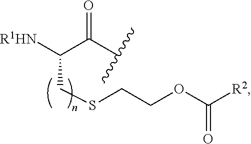
wherein n is 1, X₁ is Dab and R² is as defined in claim 1.

3. A compound of claim 1 wherein A is

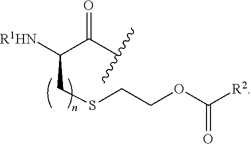

4. (canceled)

5. A compound of claim 1 wherein A is A2, n is 1 and R² is as defined in claim 1.

6. A compound of claim 1 wherein A is

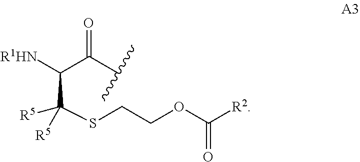

7. A compounds of claim 1 wherein R¹ is H.

8. A compound of claim 1 wherein R² is selected from the group consisting of —(C₂-C₁₀)alkyl, —(C₃-C₁₀)cycloalkyl, aryl, aryl(C₁-C₁₀)alkyl and —(C₁-C₁₀)alkylaryl, preferably —(C₂-C₁₀)alkyl, aryl and aryl(C₁-C₁₀)alkyl.

9. A compound of claim 1 wherein X₃ is Dab.

10. A compound of claim 1 wherein X₄ is Lys or Dab.

11. A compound of claim 1 wherein X₆ is D-Phe.

12. A compound of claim 1 wherein X₇ is Leu.

13. A compound of claim 1 wherein A is A1 wherein n is 1, and R² is selected from the group consisting of —(C₂-C₁₀)alkyl, aryl and aryl(C₁-C₁₀)alkyl, R¹ is H or —C(O)—(C₁-C₁₀)alkyl, X₁ is Dab, X₂ is Thr, X₃ is Dab, X₄ is Dab or Lys, X₆ is D-Phe or 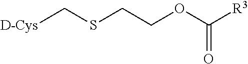
wherein R³ is selected from the group consisting of —(C₂-C₁₀)alkyl, —(C₃-C₁₀)cycloalkyl, aryl, aryl(C₁-C₁₀)alkyl and —(C₁-C₁₀)alkylaryl, preferably —(C₂-C₁₀)alkyl, aryl and aryl(C₁-C₁₀)alkyl, and X₇ is Leu or 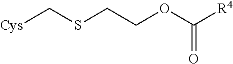
wherein R⁴ is selected from the group consisting of —(C₂-C₁₀)alkyl, —(C₃-C₁₀)cycloalkyl, aryl, aryl(C₁-C₁₀)alkyl and —(C₁-C₁₀)alkylaryl, preferably —(C₂-C₁₀)alkyl, aryl and aryl(C₁-C₁₀)alkyl.

14. A compound of claim 1 wherein A is A1 wherein n is 1, and R² is selected from the group consisting of —(C₂-C₁₀)alkyl, aryl and aryl(C₁-C₁₀)alkyl, R¹ is H, X₁ is absent, X₃ is Dab, Dap or D-Ser, X₄ is Dab, X₆ is D-Phe and X₇ is Leu.

15. A compound of claim 1 wherein A is A2 wherein n is 1, and R² is selected from the group consisting of —(C₂-C₁₀)alkyl, aryl and aryl(C₁-C₁₀)alkyl, R¹ is H, X₁ is Dab, X₃ is Dab, X₄ is Dab or Lys, X₆ is D-Phe and X₇ is Leu.

16. A compound of claim 1 wherein A is A1 wherein n is 2, and R² is selected from the group consisting of —(C₂-C₁₀)alkyl, aryl and aryl(C₁-C₁₀)alkyl, R¹ is H, X₁ is Dab, X₃ is Dab, X₄ is Dab or Lys, X₆ is D-Phe and X₇ is Leu.

17. A compound of claim 1 wherein the compound is selected from the group consisting of the polymyxin analogues defined in Table 1 or Table 3.

18. A compound of claim 1 which is selected from the group consisting of compounds 29, 44, 59, 60, 76 and 79: 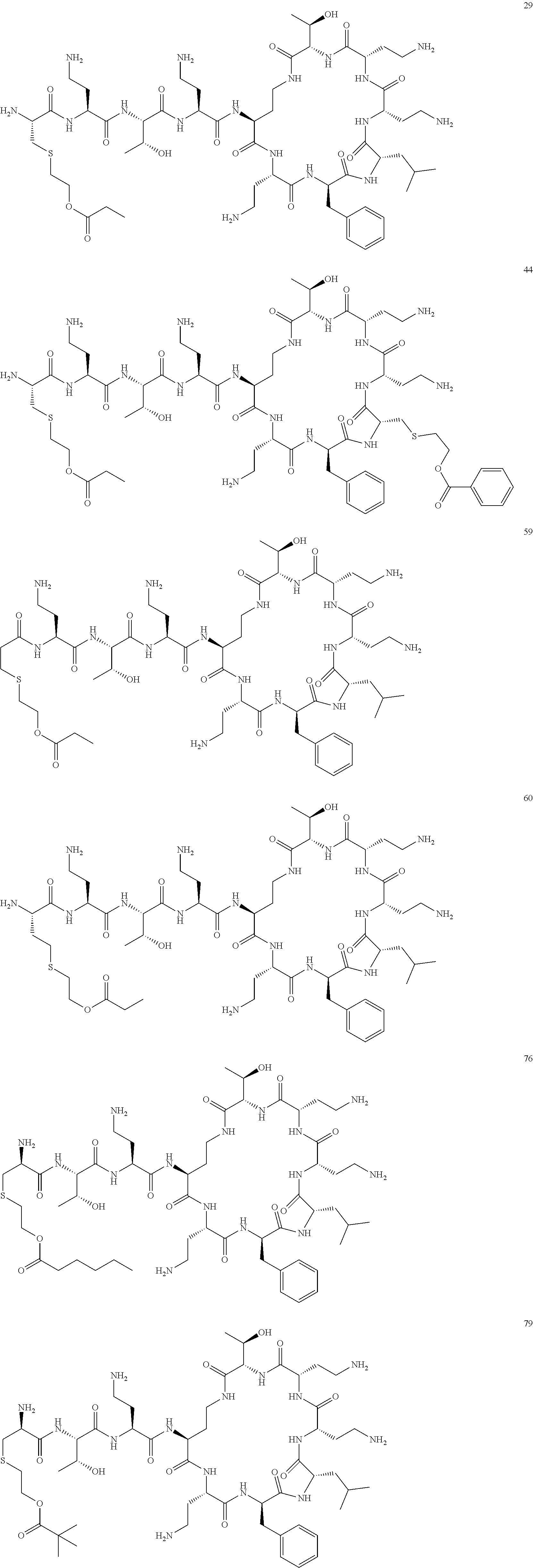
or a pharmaceutically acceptable salt or solvate thereof.

19. A pharmaceutical composition comprising a compound of claim 1 or a pharmaceutically acceptable salt or solvate thereof, and a pharmaceutically acceptable carrier.

20. (canceled)

21. A method of killing bacteria comprising contacting the bacteria with a bactericidal amount of a compound of claim 1 or a pharmaceutically acceptable salt or solvate thereof.

22-27. (canceled)
